(19) United States
(12) Patent Application Publication
(10) Pub. No.: US 2025/0122574 A1
(43) Pub. Date: Apr. 17, 2025

(54) Piezo Type Mechanosensitive Ion Channel Component 1 (PIEZO1) Variants And Uses Thereof

(71) Applicant: Regeneron Pharmaceuticals, Inc., Tarrytown, NY (US)

(72) Inventors: Joshua Backman, Tarrytown, NY (US); Aris Baras, Tarrytown, NY (US)

(21) Appl. No.: 18/933,090

(22) Filed: Oct. 31, 2024

Related U.S. Application Data
(63) Continuation of application No. 18/096,232, filed on Jan. 12, 2023, now Pat. No. 12,163,192.
Application No. 18/096,232 is a continuation of application No. 16/785,152, filed on Feb. 7, 2020, now Pat. No. 11,584,966.
(60) Provisional application No. 62/862,847, filed on Jun. 18, 2019.
(60) Provisional application No. 62/806,932, filed on Feb. 18, 2019.

(51) Int. Cl. C12Q 1/6883; A61K 31/192; A61K 31/616; A61P 9/14; C12N 15/10; C12Q 1/6827

(52) U.S. Cl. CPC C12Q 1/6883; A61K 31/192; A61K 31/616; A61P 9/14; C12N 15/1096; C12Q 1/6827; C12Q 2600/156

(57) ABSTRACT

Methods of treating patients having varicose veins, methods of identifying subjects having an increased risk of developing varicose veins, and methods of diagnosing varicose veins in a human subject, comprising detecting the presence of Piezo Type Mechanosensitive Ion Channel Component 1 (PIEZO1) predicted loss-of-function variant nucleic acid molecules and polypeptides in a biological sample from the patient or subject, are provided herein.

### REFERENCE TO A SEQUENCE LISTING

This application includes a Sequence Listing submitted electronically as an XML file named 381204294SEQ, created on Aug. 5, 2024, with a size of 93,075 bytes. The Sequence Listing is incorporated by reference herein.

### FIELD

The present disclosure provides methods of treating patients having varicose veins, methods of identifying subjects having an increased risk of developing varicose veins, and methods of diagnosing varicose veins in a human subject, comprising detecting the presence of PIEZO1 predicted loss-of-function variant nucleic acid molecules and polypeptides in a biological sample from the patient or subject.

### BACKGROUND

Varicose veins is a common multifactorial disease with largely unknown genetic drivers that is often seen in patients with chronic venous insufficiency, together classified as chronic venous disease. Dysfunction of venous valves is associated with varicose veins, venous hypertension, and thrombosis. Several processes, such as changes in hemodynamic forces, endothelial activation, inflammation, hypoxia, and dysregulation of matrix metalloproteinases and their tissue inhibitors have been associated with varicose vein development. Varicose vein risk factors include increased age, female sex, number of pregnancies, obesity, history of deep venous thrombosis, and standing occupation. Varicose veins has also been linked to insufficient lymph drainage and chronic venous insufficiency. In addition, several genome-wide association studies (GWAS) displayed about 18.5% varicose vein heritability.

PIEZO1 is encoded by a 70 kb gene located at 16q24.3 and is present in five potential isoforms. PIEZO1 protein is 2,521 amino acids long, and is a 286 kDa transmembrane protein that contains 38 transmembrane domains and functions as a homo-tetramer. PIEZO1 encodes an evolutionarily conserved endothelial mechanosensitive cation channel, which generates currents characterized by a linear current-voltage relationship that are sensitive to ruthenium red and gadolinium. PIEZO1 is ubiquitously expressed and plays a role in epithelial cell adhesion by maintaining integrin activation through R-Ras recruitment to the endoplasmic reticulum, most probably in its activated state, and subsequent stimulation of calpain signaling. In vasculature, PIEZO1 is involved in endothelial cell migration and sprouting angiogenesis. Specifically, PIEZO1 acts as a sensor for bloodflow-associated shear stress and promotes endothelial cell organization and alignment in the direction of blood flow ensuring proper vessel formation, remodeling, and maturation. PIEZO1 also appears to be required for lymphatic valve formation. Other reported functions include blood pressure regulation, urine osmolarity, erythrocyte integrity, pressure sensing, and collecting duct osmoregulation.

### SUMMARY

The present disclosure provides methods of identifying a human subject having an increased risk of developing varicose veins, wherein the method comprises determining or having determined in a biological sample obtained from the subject the presence or absence of: a Piezo Type Mechanosensitive Ion Channel Component 1 (PIEZO1) predicted loss-of-function variant genomic nucleic acid molecule; a PIEZO1 predicted loss-of-function variant mRNA molecule; a PIEZO1 predicted loss-of-function variant cDNA molecule produced from the mRNA molecule; or a PIEZO1 predicted loss-of-function variant polypeptide; wherein: the absence of the PIEZO1 predicted loss-of-function variant genomic nucleic acid molecule, mRNA molecule, cDNA molecule, or polypeptide indicates that the subject does not have an increased risk for developing varicose veins; and the presence of the PIEZO1 predicted loss-of-function variant genomic nucleic acid molecule, mRNA molecule, cDNA molecule, or polypeptide indicates that the subject has an increased risk for developing varicose veins.

The present disclosure also provides methods of diagnosing varicose veins in a human subject, wherein the method comprises detecting in a sample obtained from the subject the presence or absence of: a Piezo Type Mechanosensitive Ion Channel Component 1 (PIEZO1) predicted loss-of-function variant genomic nucleic acid molecule; a PIEZO1 predicted loss-of-function variant mRNA molecule; a PIEZO1 predicted loss-of-function variant cDNA molecule produced from the mRNA molecule; or a PIEZO1 predicted loss-of-function variant polypeptide; wherein when the subject has a PIEZO1 predicted loss-of-function variant genomic nucleic acid molecule, mRNA molecule, cDNA molecule, or polypeptide, and has one or more symptoms of varicose veins, then the subject is diagnosed as having varicose veins.

The present disclosure also provides methods of treating a patient with a therapeutic agent that treats or inhibits varicose veins, wherein the patient is suffering from varicose veins or has an increased risk of developing varicose veins, the method comprising the steps of: determining whether the patient has a Piezo Type Mechanosensitive Ion Channel Component 1 (PIEZO1) predicted loss-of-function variant nucleic acid molecule encoding a human PIEZO1 polypeptide by: obtaining or having obtained a biological sample from the patient; and performing or having performed a genotyping assay on the biological sample to determine if the patient has a genotype comprising the PIEZO1 predicted loss-of-function variant nucleic acid molecule; and when the patient is PIEZO1 reference, then administering or continuing to administer to the patient the therapeutic agent that treats or inhibits the varicose veins in a standard dosage amount; and when the patient is heterozygous or homozygous for a PIEZO1 predicted loss-of-function variant nucleic acid molecule, then administering or continuing to administer to the patient the therapeutic agent that treats or inhibits the varicose veins in an amount that is the same as or greater than the standard dosage amount; wherein the presence of a genotype having the PIEZO1 predicted loss-of-function variant nucleic acid molecule encoding the human PIEZO1 polypeptide indicates the patient has an increased risk of developing varicose veins.

### BRIEF DESCRIPTION OF THE FIGURES

The accompanying figures, which are incorporated in and constitute a part of this specification, illustrate several aspects and together with the description serve to explain the principles of the present disclosure.

FIG. 1 shows a representative distribution of IBD sharing for pairs of individuals in UKB 50 k WES; estimated proportion of WES genotypes with no alleles identical by descent (IBD) vs. 1 allele IBD amongst all pairs of UKB 50 k exome participants.

FIG. 2 shows an observed site frequency spectrum (SFS) for all autosomal variants and by functional prediction; UKB 50 k exomes were down-sampled at random to the number of individuals specified on the horizontal axis; the number of genes containing at least the indicated count of LOFs AAF<1% as in the legend are plotted on the vertical axis; the maximum number of autosomal genes is 18,272.

FIG. 3 shows continental ancestry in UK Biobank 500 k and 50 k; principal component 1 and 2 for n=488,377 individuals available from the UK Biobank Data Showcase; three pre-defined regions of a plot of represent African (blue), East Asian (green), and European (red) ancestry.

FIG. 4 shows Single-point and aggregate results for the 65 pLOF variants in PIEZO1.

FIG. 5 shows LD Assessment for PIEZO1.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the terms “subject” and “patient” are used interchangeably. A subject may include any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human.

As used herein, a “nucleic acid,” a “nucleic acid molecule,” a “nucleic acid sequence,” a “polynucleotide,” or an “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “comprising” may be replaced with “consisting” or “consisting essentially of” in particular embodiments as desired.

As used herein, the phrase “corresponding to” or grammatical variations thereof when used in the context of the numbering of a particular amino acid or nucleotide sequence or position refers to the numbering of a specified reference sequence when the particular amino acid or nucleotide sequence is compared to the reference sequence (e.g., with the reference sequence herein being the nucleic acid molecule or polypeptide of (wild type) PIEZO1). In other words, the residue (e.g., amino acid or nucleotide) number or residue (e.g., amino acid or nucleotide) position of a particular polymer is designated with respect to the reference sequence rather than by the actual numerical position of the residue within the particular amino acid or nucleotide sequence. For example, a particular amino acid sequence can be aligned to a reference sequence by introducing gaps to optimize residue matches between the two sequences. In these cases, although the gaps are present, the numbering of the residue in the particular amino acid or nucleotide sequence is made with respect to the reference sequence to which it has been aligned.

It has been observed in accordance with the present disclosure that certain variations in PIEZO1 associate with a risk of developing varicose veins. It is believed that no variants of the PIEZO1 gene or protein have any known association with varicose veins in human beings. Therefore, human subjects having PIEZO1 alterations that associate with varicose veins may be treated such that varicose veins is inhibited, the symptoms thereof are reduced, and/or development of symptoms is repressed. Accordingly, the present disclosure provides methods for leveraging the identification of such variants in subjects to identify or stratify risk in such subjects of developing varicose veins, or to diagnose subjects as having varicose veins, such that subjects at risk or subjects with active disease may be treated.

For purposes of the present disclosure, any particular human can be categorized as having one of three PIEZO1 genotypes: i) PIEZO1 reference; ii) heterozygous for a PIEZO1 predicted loss-of-function variant, and iii) homozygous for a PIEZO1 predicted loss-of-function variant. A human is PIEZO1 reference when the human does not have a copy of a PIEZO1 predicted loss-of-function variant nucleic acid molecule. A human is heterozygous for a PIEZO1 predicted loss-of-function variant when the human has a single copy of a PIEZO1 predicted loss-of-function variant nucleic acid molecule. A PIEZO1 predicted loss-of-function variant nucleic acid molecule is any PIEZO1 nucleic acid molecule (such as, a genomic nucleic acid molecule, an mRNA molecule, or a cDNA molecule) encoding a PIEZO1 polypeptide having a partial loss-of-function, a complete loss-of-function, a predicted partial loss-of-function, or a predicted complete loss-of-function. A human who has a PIEZO1 polypeptide having a partial loss-of-function (or predicted partial loss-of-function) is hypomorphic for PIEZO1. The PIEZO1 predicted loss-of-function variant nucleic acid molecule can be any variant nucleic acid molecule described herein. A human is homozygous for a PIEZO1 predicted loss-of-function variant when the human has two copies of any of the PIEZO1 predicted loss-of-function variant nucleic acid molecules.

For human subjects or patients that are genotyped or determined to be heterozygous or homozygous for a PIEZO1 predicted loss-of-function variant nucleic acid molecule, such human subjects or patients have an increased risk of developing varicose veins. For human subjects or patients that are genotyped or determined to be heterozygous or homozygous for a PIEZO1 predicted loss-of-function variant nucleic acid molecule, such human subjects or patients can be treated with an agent effective to treat varicose veins.

The present disclosure provides methods of identifying a human subject having an increased risk of developing varicose veins, wherein the method comprises determining or having determined in a biological sample obtained from the subject the presence or absence of a PIEZO1 predicted loss-of-function variant nucleic acid molecule (such as a genomic nucleic acid molecule, mRNA molecule, and/or cDNA molecule) or polypeptide; wherein the absence of the PIEZO1 predicted loss-of-function variant nucleic acid molecule or polypeptide indicates that the subject does not have an increased risk for developing varicose veins; and the presence of the PIEZO1 predicted loss-of-function variant genomic nucleic acid molecule, mRNA molecule, cDNA molecule, or polypeptide indicates that the subject has an increased risk for developing varicose veins.

The present disclosure also provides methods of identifying a human subject having an increased risk of developing varicose veins, wherein the method comprises determining or having determined in a biological sample obtained from the subject the presence or absence of: i) a PIEZO1 predicted loss-of-function variant genomic nucleic acid molecule; ii) a PIEZO1 predicted loss-of-function variant mRNA molecule; iii) a PIEZO1 predicted loss-of-function variant cDNA molecule produced from the mRNA molecule; or iv) a PIEZO1 predicted loss-of-function variant polypeptide; wherein: the absence of the PIEZO1 predicted loss-of-function variant genomic nucleic acid molecule, mRNA molecule, cDNA molecule, or polypeptide indicates that the subject does not have an increased risk for developing varicose veins; and the presence of the PIEZO1 predicted loss-of-function variant genomic nucleic acid molecule, mRNA molecule, cDNA molecule, or polypeptide indicates that the subject has an increased risk for developing varicose veins.

The present disclosure also provides methods of identifying a human subject having an increased risk for developing varicose veins, wherein the method comprises: determining or having determined in a biological sample obtained from the subject the presence or absence of a PIEZO1 predicted loss-of-function variant nucleic acid molecule (such as a genomic nucleic acid molecule, mRNA molecule, and/or cDNA molecule) encoding a human PIEZO1 polypeptide; wherein: i) when the human subject lacks a PIEZO1 predicted loss-of-function variant nucleic acid molecule (i.e., the human subject is genotypically categorized as a PIEZO1 reference), then the human subject does not have an increased risk for developing varicose veins; and ii) when the human subject has a PIEZO1 predicted loss-of-function variant nucleic acid molecule (i.e., the human subject is categorized as heterozygous for a PIEZO1 predicted loss-of-function variant or homozygous for a PIEZO1 predicted loss-of-function variant), then the human subject has an increased risk for developing varicose veins.

In any of the embodiments described herein, the PIEZO1 predicted loss-of-function variant nucleic acid molecule can be any PIEZO1 nucleic acid molecule (such as, for example, genomic nucleic acid molecule, mRNA molecule, or cDNA molecule) encoding a PIEZO1 polypeptide having a partial loss-of-function, a complete loss-of-function, a predicted partial loss-of-function, or a predicted complete loss-of-function. For example, the PIEZO1 predicted loss-of-function variant nucleic acid molecule can be any of the PIEZO1 variant nucleic acid molecules described herein.

Determining whether a human subject has a PIEZO1 predicted loss-of-function variant nucleic acid molecule in a biological sample can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the human subject.

In any of the embodiments described herein, the varicose veins can be early stage varicose veins (e.g., C0 according to CEAP (Clinical, Etiological, Anatomical, and Pathophysiological) classification). In some embodiments, the varicose veins can be late stage varicose veins (e.g., C6 according to CEAP classification). In some embodiments, the varicose veins can be at any disease stage (e.g., C0-C6 according to CEAP classification). In some embodiments, the human subject is a female.

In some embodiments, when a human subject is identified as having an increased risk of developing varicose veins, the human subject is further treated with a therapeutic agent that treats or inhibits varicose veins, as described herein. For example, when the human subject is heterozygous or homozygous for a PIEZO1 predicted loss-of-function variant nucleic acid molecule, and therefore has an increased risk for developing varicose veins, the human subject is administered a therapeutic agent that treats or inhibits varicose veins. In some embodiments, when the patient is homozygous for a PIEZO1 predicted loss-of-function variant nucleic acid molecule, the patient is administered the therapeutic agent that treats or inhibits varicose veins in a dosage amount that is the same as or greater than the standard dosage amount administered to a patient who is heterozygous for a PIEZO1 predicted loss-of-function variant nucleic acid molecule. In some embodiments, the patient is heterozygous for a PIEZO1 predicted loss-of-function variant nucleic acid molecule. In some embodiments, the patient is homozygous for a PIEZO1 predicted loss-of-function variant nucleic acid molecule.

The present disclosure provides methods of diagnosing varicose veins in a human subject, wherein the methods comprise detecting in a sample obtained from the subject the presence or absence of a PIEZO1 predicted loss-of-function variant nucleic acid molecule (such as a genomic nucleic acid molecule, mRNA molecule, and/or cDNA molecule) or polypeptide; wherein when the subject has a PIEZO1 predicted loss-of-function variant genomic nucleic acid molecule, mRNA molecule, cDNA molecule, or polypeptide, and has one or more symptoms of varicose veins, then the subject is diagnosed as having varicose veins.

The present disclosure also provides methods of diagnosing varicose veins in a human subject, wherein the methods comprise detecting in a sample obtained from the subject the presence or absence of: i) a PIEZO1 predicted loss-of-function variant genomic nucleic acid molecule; ii) a PIEZO1 predicted loss-of-function variant mRNA molecule; iii) a PIEZO1 predicted loss-of-function variant cDNA molecule produced from the mRNA molecule; or iv) a PIEZO1 predicted loss-of-function variant polypeptide; wherein when the subject has a PIEZO1 predicted loss-of-function variant genomic nucleic acid molecule, mRNA molecule, cDNA molecule, or polypeptide, and has one or more symptoms of varicose veins, then the subject is diagnosed as having varicose veins.

The present disclosure also provides methods of diagnosing varicose veins in a human subject, wherein the methods comprise detecting in a sample obtained from the subject the presence or absence of a PIEZO1 predicted loss-of-function variant nucleic acid molecule (such as a genomic nucleic acid molecule, mRNA molecule, and/or cDNA molecule) encoding a human PIEZO1 polypeptide; wherein when the subject has a PIEZO1 predicted loss-of-function variant genomic nucleic acid molecule, mRNA molecule, cDNA molecule, or polypeptide (i.e., the human subject is categorized as heterozygous or homozygous for a PIEZO1 predicted loss-of-function variant nucleic acid molecule), and has one or more symptoms of varicose veins, then the subject is diagnosed as having varicose veins

In any of the embodiments described herein, the PIEZO1 predicted loss-of-function variant nucleic acid molecule can be any PIEZO1 nucleic acid molecule (such as, for example, genomic nucleic acid molecule, mRNA molecule, or cDNA molecule) encoding a PIEZO1 polypeptide having a partial loss-of-function, a complete loss-of-function, a predicted partial loss-of-function, or a predicted complete loss-of-function. For example, the PIEZO1 predicted loss-of-function variant nucleic acid molecule can be any of the PIEZO1 variant nucleic acid molecules described herein.

Detecting the presence or absence of a PIEZO1 predicted loss-of-function variant nucleic acid molecule in a sample obtained from the subject can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the human subject.

In any of the embodiments described herein, the varicose veins can be early stage varicose veins (e.g., C0 according to CEAP (Clinical, Etiological, Anatomical, and

Pathophysiological) classification). In some embodiments, the varicose veins can be late stage varicose veins (e.g., C6 according to CEAP classification). In some embodiments, the varicose veins can be at any disease stage (e.g., C0-C6 according to CEAP classification). In some embodiments, the human subject is a female.

In some embodiments, when a human subject is diagnosed as having varicose veins, the human subject is further treated with a therapeutic agent that treats or inhibits varicose veins, as described herein. For example, when the human subject is determined to be heterozygous or homozygous for a PIEZO1 predicted loss-of-function variant nucleic acid molecule, and has one or more symptoms of varicose veins, the human subject is administered a therapeutic agent that treats or inhibits varicose veins. In some embodiments, when the patient is homozygous for a PIEZO1 predicted loss-of-function variant nucleic acid molecule, the patient is administered the therapeutic agent that treats or inhibits varicose veins in a dosage amount that is the same as or greater than the standard dosage amount administered to a patient who is heterozygous for a PIEZO1 predicted loss-of-function variant nucleic acid molecule. In some embodiments, the patient is heterozygous for a PIEZO1 predicted loss-of-function variant nucleic acid molecule. In some embodiments, the patient is homozygous for a PIEZO1 predicted loss-of-function variant nucleic acid molecule.

The present disclosure also provides methods of treating a patient with a therapeutic agent that treats or inhibits varicose veins, wherein the patient is suffering from varicose veins or has an increased risk of developing varicose veins, the methods comprising the steps of: determining whether the patient has a PIEZO1 predicted loss-of-function variant nucleic acid molecule encoding a human PIEZO1 polypeptide by: obtaining or having obtained a biological sample from the patient; and performing or having performed a genotyping assay on the biological sample to determine if the patient has a genotype comprising the PIEZO1 predicted loss-of-function variant nucleic acid molecule; and when the patient is PIEZO1 reference, then administering or continuing to administer to the patient the therapeutic agent that treats or inhibits the varicose veins in a standard dosage amount; and when the patient is heterozygous or homozygous for a PIEZO1 predicted loss-of-function variant nucleic acid molecule, then administering or continuing to administer to the patient the therapeutic agent that treats or inhibits the varicose veins in an amount that is the same as or greater than the standard dosage amount; wherein the presence of a genotype having the PIEZO1 predicted loss-of-function variant nucleic acid molecule encoding the human PIEZO1 polypeptide indicates the patient has an increased risk of developing varicose veins. In some embodiments, the patient is heterozygous for a PIEZO1 predicted loss-of-function variant nucleic acid molecule. In some embodiments, the patient is homozygous for a PIEZO1 predicted loss-of-function variant nucleic acid molecule.

In any of the embodiments described herein, the PIEZO1 predicted loss-of-function variant nucleic acid molecule can be any PIEZO1 nucleic acid molecule (such as, for example, genomic nucleic acid molecule, mRNA molecule, or cDNA molecule) encoding a PIEZO1 polypeptide having a partial loss-of-function, a complete loss-of-function, a predicted partial loss-of-function, or a predicted complete loss-of-function. For example, the PIEZO1 predicted loss-of-function variant nucleic acid molecule can be any of the PIEZO1 variant nucleic acid molecules described herein.

The genotyping assay to determine whether a patient has a PIEZO1 predicted loss-of-function variant nucleic acid molecule encoding a human PIEZO1 polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the human subject.

In some embodiments, when the patient is homozygous for a PIEZO1 predicted loss-of-function variant nucleic acid molecule, the patient is administered the therapeutic agent that treats or inhibits varicose veins in a dosage amount that is the same as or greater than the standard dosage amount administered to a patient who is heterozygous for a PIEZO1 predicted loss-of-function variant nucleic acid molecule.

The present disclosure also provides methods of treating a patient with a therapeutic agent that treats or inhibits varicose veins, wherein the patient is suffering from varicose veins or has an increased risk of developing varicose veins, the methods comprising the steps of: determining whether the patient has a PIEZO1 predicted loss-of-function variant polypeptide by: obtaining or having obtained a biological sample from the patient; and performing or having performed an assay on the biological sample to determine if the patient has a PIEZO1 predicted loss-of-function variant polypeptide; and when the patient does not have a PIEZO1 predicted loss-of-function variant polypeptide, then administering or continuing to administer to the patient the therapeutic agent that treats or inhibits the varicose veins in a standard dosage amount; and when the patient has a PIEZO1 predicted loss-of-function variant polypeptide, then administering or continuing to administer to the patient the therapeutic agent that treats or inhibits the varicose veins in an amount that is the same as or greater than the standard dosage amount; wherein the presence of a PIEZO1 predicted loss-of-function variant polypeptide indicates the patient has an increased risk of developing varicose veins. In some embodiments, the patient has a PIEZO1 predicted loss-of-function variant polypeptide. In some embodiments, the patient does not have a PIEZO1 predicted loss-of-function variant polypeptide.

The assay to determine whether a patient has a PIEZO1 predicted loss-of-function variant polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the human subject.

In any of the embodiments described herein, the PIEZO1 predicted loss-of-function variant polypeptide can be any PIEZO1 polypeptide having a partial loss-of-function, a complete loss-of-function, a predicted partial loss-of-function, or a predicted complete loss-of-function. For example, the PIEZO1 predicted loss-of-function variant polypeptide can be any of the PIEZO1 variant polypeptides described herein.

In any of the embodiments described herein, the varicose veins can be early stage varicose veins (e.g., C0 according to CEAP (Clinical, Etiological, Anatomical, and Pathophysiological) classification). In some embodiments, the varicose veins can be late stage varicose veins (e.g., C6 according to CEAP classification). In some embodiments, the varicose veins can be at any disease stage (e.g., C0-C6 according to CEAP classification). In some embodiments, the human subject is a female.

Symptoms of varicose veins include, but are not limited to, heavy legs, appearance of spider veins (telangiectasia) in the affected leg, ankle swelling, especially in the evening, brownish-yellow shiny skin discoloration near the affected veins, redness, dryness, and itchiness of areas of skin (termed stasis dermatitis or venous eczema), cramps especially developing when making a sudden move such as standing up, minor injuries to the affected area, bleeding more than normal or taking a long time to heal, shrinking of the skin above the ankle (lipodermatosclerosis), restless legs syndrome, whitened, irregular scar-like patches appearing at the ankles (atrophie blanche), or any combination thereof.

Examples of therapeutic agents that treat or inhibit varicose veins include, but are not limited to flavonoids, such as diosmin or hesperidin, and anti-inflammatory agents, such as ibuprofen and aspirin.

In some embodiments, the dose of the therapeutic agents that treat or inhibit varicose veins can be reduced by about 10%, by about 20%, by about 30%, by about 40%, by about 50%, by about 60%, by about 70%, by about 80%, or by about 90% for patients or human subjects that are heterozygous for a PIEZO1 predicted loss-of-function variant nucleic acid molecule (i.e., a lower than the standard dosage amount) compared to patients or human subjects that are homozygous for a PIEZO1 predicted loss-of-function variant nucleic acid molecule (who may receive a standard dosage amount). In some embodiments, the dose of the therapeutic agents that treat or inhibit varicose veins can be reduced by about 10%, by about 20%, by about 30%, by about 40%, or by about 50%. In addition, the dose of therapeutic agents that treat or inhibit varicose veins in patients or human subjects that are heterozygous for a PIEZO1 predicted loss-of-function variant nucleic acid molecule can be administered less frequently compared to patients or human subjects that are homozygous for a PIEZO1 predicted loss-of-function variant nucleic acid molecule.

Administration of the therapeutic agents that treat or inhibit varicose veins can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a patient can receive therapy for a prolonged period of time such as, for example, 6 months, 1 year, or more.

Administration of the therapeutic agents that treat or inhibit varicose veins can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in varicose veins, a decrease/reduction in the severity of varicose veins (such as, for example, a reduction or inhibition of development of varicose veins), a decrease/reduction in symptoms and varicose vein-related effects, delaying the onset of symptoms and varicose vein-related effects, reducing the severity of symptoms of varicose vein-related effects, reducing the severity of an acute episode, reducing the number of symptoms and varicose vein-related effects, reducing the latency of symptoms and varicose vein-related effects, an amelioration of symptoms and varicose vein-related effects, reducing secondary symptoms, reducing secondary infections, preventing relapse to varicose veins, decreasing the number or frequency of relapse episodes, increasing latency between symptomatic episodes, increasing time to sustained progression, speeding recovery, and/or increasing efficacy of or decreasing resistance to alternative therapeutics, following administration of the agent or composition comprising the agent. A prophylactic effect may comprise a complete or partial avoidance/inhibition or a delay of varicose vein development/progression (such as, for example, a complete or partial avoidance/inhibition or a delay) following administration of a therapeutic protocol. Treatment of varicose veins encompasses the treatment of patients already diagnosed as having any form of varicose veins at any clinical stage or manifestation, the delay of the onset or evolution or aggravation or deterioration of the symptoms or signs of varicose veins, and/or preventing and/or reducing the severity of varicose veins.

The present disclosure also provides, in any of the methods described herein, the detection or determination of the presence of a PIEZO1 predicted loss-of-function variant genomic nucleic acid molecule, a PIEZO1 predicted loss-of-function variant mRNA molecule, and/or a PIEZO1 predicted loss-of-function variant cDNA molecule in a biological sample from a subject human. It is understood that gene sequences within a population and mRNA molecules encoded by such genes can vary due to polymorphisms such as single-nucleotide polymorphisms. The sequences provided herein for the PIEZO1 variant nucleic acid molecules disclosed herein are only exemplary sequences. Other sequences for the PIEZO1 variant nucleic acid molecules are also possible.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The sample used in the methods disclosed herein will vary based on the assay format, nature of the detection method, and the tissues, cells, or extracts that are used as the sample. A biological sample can be processed differently depending on the assay being employed. For example, when detecting any PIEZO1 variant nucleic acid molecule, preliminary processing designed to isolate or enrich the sample for the genomic DNA can be employed. A variety of known techniques may be used for this purpose. When detecting the level of any PIEZO1 variant mRNA, different techniques can be used enrich the biological sample with mRNA. Various methods to detect the presence or level of a mRNA or the presence of a particular variant genomic DNA locus can be used.

In some embodiments, detecting a human PIEZO1 predicted loss-of-function variant nucleic acid molecule in a human subject comprises assaying or genotyping a biological sample obtained from the human subject to determine whether a PIEZO1 genomic nucleic acid molecule, a PIEZO1 mRNA molecule, or a PIEZO1 cDNA molecule produced from an mRNA molecule in the biological sample comprises one or more variations that cause a loss-of-function (partial or complete) or are predicted to cause a loss-of-function (partial or complete).

In some embodiments, the methods of detecting the presence or absence of a PIEZO1 predicted loss-of-function variant nucleic acid molecule (such as, for example, a genomic nucleic acid molecule, an mRNA molecule, and/or a cDNA molecule) in a human subject, comprise: performing an assay on a biological sample obtained from the human subject, which assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising a PIEZO1 genomic nucleic acid molecule or mRNA molecule, and if mRNA, optionally reverse transcribing the mRNA into cDNA. Such assays can comprise, for example determining the identity of these positions of the particular PIEZO1 nucleic acid molecule. In some embodiments, the method is an in vitro method.

In some embodiments, the determining step, detecting step, or genotyping assay comprises sequencing at least a portion of the nucleotide sequence of the PIEZO1 genomic nucleic acid molecule, the PIEZO1 mRNA molecule, or the PIEZO1 cDNA molecule produced from the mRNA molecule in the biological sample, wherein the sequenced portion comprises one or more variations that cause a loss-of-function (partial or complete) or are predicted to cause a loss-of-function (partial or complete).

In any of the methods described herein, the determining step, detecting step, or genotyping assay comprises sequencing at least a portion of the nucleotide sequence of the PIEZO1 nucleic acid molecule in the biological sample, wherein the sequenced portion comprises a position corresponding to a predicted loss-of-function variant position, wherein when a variant nucleotide at the predicted loss-of-function variant position is detected, the PIEZO1 nucleic acid molecule in the biological sample is a PIEZO1 predicted loss-of-function variant nucleic acid molecule.

In some embodiments, the determining step, detecting step, or genotyping assay comprises: a) contacting the biological sample with a primer hybridizing to a portion of the nucleotide sequence of the PIEZO1 nucleic acid molecule that is proximate to a predicted loss-of-function variant position; b) extending the primer at least through the predicted loss-of-function variant position; and c) determining whether the extension product of the primer comprises a variant nucleotide at the predicted loss-of-function variant position.

In some embodiments, the assay comprises sequencing the entire nucleic acid molecule. In some embodiments, only a PIEZO1 genomic nucleic acid molecule is analyzed. In some embodiments, only a PIEZO1 mRNA is analyzed. In some embodiments, only a PIEZO1 cDNA obtained from PIEZO1 mRNA is analyzed.

In some embodiments, the determining step, detecting step, or genotyping assay comprises: a) amplifying at least a portion of the PIEZO1 nucleic acid molecule that encodes the human PIEZO1 polypeptide, wherein the portion comprises a predicted loss-of-function variant position; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the predicted loss-of-function variant position; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to a predicted loss-of-function variant position; and detecting the detectable label.

The alteration-specific probes or alteration-specific primers described herein comprise a nucleic acid sequence which is complementary to and/or hybridizes, or specifically hybridizes, to a PIEZO1 predicted loss-of-function variant nucleic acid molecule, or the complement thereof. In some embodiments, the alteration-specific probes or alteration-specific primers comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, or at least about 50 nucleotides. In some embodiments, the alteration-specific probes or alteration-specific primers comprise or consist of at least 15 nucleotides. In some embodiments, the alteration-specific probes or alteration-specific primers comprise or consist of at least 15 nucleotides to at least about 35 nucleotides. In some embodiments, alteration-specific probes or alteration-specific primers hybridize to PIEZO1 predicted loss-of-function variant genomic nucleic acid molecules, PIEZO1 predicted loss-of-function variant mRNA molecules, and/or PIEZO1 predicted loss-of-function variant cDNA molecules under stringent conditions.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the human subject.

In any of the embodiments described herein, the PIEZO1 predicted loss-of-function variant nucleic acid molecule can be any PIEZO1 nucleic acid molecule (such as, for example, genomic nucleic acid molecule, mRNA molecule, or cDNA molecule) encoding a PIEZO1 polypeptide having a partial loss-of-function, a complete loss-of-function, a predicted partial loss-of-function, or a predicted complete loss-of-function. For example, the PIEZO1 predicted loss-of-function variant nucleic acid molecule can be any of the PIEZO1 variant nucleic acid molecules described herein.

In some embodiments, the assay comprises contacting the biological sample with a primer or probe, such as an alteration-specific primer or alteration-specific probe, that specifically hybridizes to a PIEZO1 variant genomic sequence, variant mRNA sequence, or variant cDNA sequence and not the corresponding PIEZO1 reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a PIEZO1 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2× SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides molecular complexes comprising any of the PIEZO1 nucleic acid molecules (genomic nucleic acid molecules, mRNA molecules, or cDNA molecules), or complement thereof, described herein and any of the alteration-specific primers or alteration-specific probes described herein. In some embodiments, the PIEZO1 nucleic acid molecules (genomic nucleic acid molecules, mRNA molecules, or cDNA molecules), or complement thereof, in the molecular complexes are single-stranded. In some embodiments, the PIEZO1 nucleic acid molecule is any of the genomic nucleic acid molecules described herein. In some embodiments, the PIEZO1 nucleic acid molecule is any of the mRNA molecules described herein. In some embodiments, the PIEZO1 nucleic acid molecule is any of the cDNA molecules described herein. In some embodiments, the molecular complex comprises any of the PIEZO1 nucleic acid molecules (genomic nucleic acid molecules, mRNA molecules, or cDNA molecules), or complement thereof, described herein and any of the alteration-specific primers described herein. In some embodiments, the molecular complex comprises any of the PIEZO1 nucleic acid molecules (genomic nucleic acid molecules, mRNA molecules, or cDNA molecules), or complement thereof, described herein and any of the alteration-specific probes described herein. In some embodiments, the molecular complex comprises a non-human polymerase.

In some embodiments, detecting the presence of a human PIEZO1 predicted loss-of-function polypeptide comprises performing an assay on a sample obtained from a human subject to determine whether a PIEZO1 polypeptide in the subject contains one or more variations that causes the polypeptide to have a loss-of-function (partial or complete) or predicted loss-of-function (partial or complete). In some embodiments, the assay comprises sequencing at least a portion of the PIEZO1 polypeptide that comprises a variant position. In some embodiments, the detecting step comprises sequencing the entire polypeptide. Identification of a variant amino acid at the variant position of the PIEZO1 polypeptide indicates that the PIEZO1 polypeptide is a PIEZO1 predicted loss-of-function polypeptide. In some embodiments, the assay comprises an immunoassay for detecting the presence of a polypeptide that comprises a variant. Detection of a variant amino acid at the variant position of the PIEZO1 polypeptide indicates that the PIEZO1 polypeptide is a PIEZO1 predicted loss-of-function polypeptide.

The probes and/or primers (including alteration-specific probes and alteration-specific primers) described herein comprise or consist of from about 15 to about 100, from about 15 to about 35 nucleotides. In some embodiments, the alteration-specific probes and alteration-specific primers comprise DNA. In some embodiments, the alteration-specific probes and alteration-specific primers comprise RNA. In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers (including alteration-specific probes and alteration-specific primers) specifically hybridize to any of the nucleic acid molecules disclosed herein under stringent conditions. In the context of the disclosure “specifically hybridizes” means that the probe or primer (including alteration-specific probes and alteration-specific primers) does not hybridize to a nucleic acid sequence encoding a PIEZO1 reference genomic nucleic acid molecule, a PIEZO1 reference mRNA molecule, and/or a PIEZO1 reference cDNA molecule. In some embodiments, the probes (such as, for example, an alteration-specific probe) comprise a label. In some embodiments, the label is a fluorescent label, a radiolabel, or biotin.

The nucleotide sequence of a PIEZO1 reference genomic nucleic acid molecule is set forth in SEQ ID NO:1, which is 69,883 nucleotides in length. The first nucleotide recited in SEQ ID NO:1 corresponds to the nucleotide at position 88,715,338 of chromosome 16 (see, hg38_knownGene_ENST00000301015.14).

Numerous variant genomic nucleic acid molecule of PIEZO1 exist, including, but not limited to (using the human genome reference build GRch38): 16:88715629:G:A, 16:88715728:G:T, 16:88715767:G:A, 16:88715802:C:A, 16:88715822:D:4, 16:88715987:I:1, 16:88716359:A:G, 16:88716570:C:T, 16:88716874:G:A, 16:88717213:T:A, 16:88719588:G:A, 16:88719722:C:G, 16:88719870:G:T, 16:88720068:D:2, 16:88720229:C:A, 16:88720248:D:4, 16:88720394:C:T, 16:88720644:D:1, 16:88720698:D:1, 16:88720698:I:1, 16:88721165:C:A, 16:88721268:D:1, 16:88721307:G:A, 16:88721586:G:C, 16:88721652:G:C, 16:88722217:C:T, 16:88722605:I:1, 16:88723005:I:7, 16:88723253:G:A, 16:88723311:C:T, 16:88725081:C:A, 16:88726282:G:A, 16:88726546:C:T, 16:88726619:G:A, 16:88726924:G:A, 16:88727038:C:T, 16:88727072:D:1, 16:88727163:G:A, 16:88731768:D:1, 16:88732334:C:G, 16:88732411:D:1, 16:88732720:D:1, 16:88733326:G:C, 16:88733337:D:4, 16:88733587:C:A, 16:88733965:D:1, 16:88734017:C:A, 16:88734042:I:1, 16:88734679:C:T, 16:88734909:I:1, 16:88736167:D:2, 16:88736324:G:A, 16:88736391:G:T, 16:88736409:C:T, 16:88736671:G:A, 16:88737557:A:C, 16:88737727:C:G, 16:88737815:C:T, 16:88738283:G:C, 16:88738637:G:A, 16:88738735:D:1, 16:88741477:C:T, 16:88742306:D:1, 16:88749399:G:A, and 16:88784929:C:T. Thus, for example, using the SEQ ID NO:1 reference genomic nucleotide sequence as a base (with the first nucleotide listed therein designated as position 88,715,338), the first listed variant (16:88715629:G:A) would have a guanine replaced with an adenine (designated the “variant nucleotide”) at position 88,715,629 (designated the “variant position”). Those variants designated as a “D” followed by a number have a deletion of the stated number of nucleotides. Those variants designated as an “I” followed by a number have an insertion of the stated number of nucleotides (any nucleotide). Any of these PIEZO1 predicted loss-of-function variant genomic nucleic acid molecules can be detected in any of the methods described herein.

The nucleotide sequence of a PIEZO1 reference mRNA molecule is set forth in SEQ ID NO: 2 (see, NCBI Reference Sequence: NM_001142864.4), which is 8,089 nucleotides in length. The variant nucleotides at their respective variant positions for the variant genomic nucleic acid molecules described herein also have corresponding variant nucleotides at their respective variant positions for the variant mRNA molecules based upon the PIEZO1 reference mRNA sequence according to SEQ ID NO:2. Any of these PIEZO1 predicted loss-of-function variant mRNA molecules can be detected in any of the methods described herein.

The nucleotide sequence of a PIEZO1 reference cDNA molecule is set forth in SEQ ID NO:3 (based upon NCBI Reference Sequence: NM_001142864.4), which is 8,089 nucleotides in length. The variant nucleotides at their respective variant positions for the variant genomic nucleic acid molecules described herein also have corresponding variant nucleotides at their respective variant positions for the variant cDNA molecules based upon the PIEZO1 reference cDNA sequence according to SEQ ID NO:3. Any of these PIEZO1 predicted loss-of-function variant cDNA molecules can be detected in any of the methods described herein.

The amino acid sequence of a PIEZO1 reference polypeptide is set forth in SEQ ID NO: 4 (see, UniProt Accession No. Q92508.4 and NCBI RefSeq accession NM_001142864.4), which is 2,521 amino acids in length. Using the translated nucleotide sequence of either the PIEZO1 mRNA or cDNA molecules, the PIEZO1 variant polypeptides having corresponding translated variant amino acids at variant positions (codons). Any of these PIEZO1 predicted loss-of-function variant polypeptides can be detected in any of the methods described herein.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence. In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned. A variety of computational algorithms exist that can be used for performing a sequence alignment to identify a nucleotide or amino acid position in one polymeric molecule that corresponds to a nucleotide or amino acid position in another polymeric molecule. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Materials and Methods

### WES Sample Preparation and Sequencing

Genomic DNA samples normalized to approximately 16 ng/μl were transferred in house from the UK Biobank in 0.5 ml 2D matrix tubes (Thermo Fisher Scientific) and stored in an automated sample biobank (LiCONiC Instruments) at −80° C. prior to sample preparation. One sample had insufficient DNA for sequencing. Exome capture was completed using a high-throughput, fully-automated approach developed in house. Briefly, DNA libraries were created by enzymatically shearing 100 ng of genomic DNA to a mean fragment size of 200 base pairs using a custom NEBNext Ultra II FS DNA library prep kit (New England Biolabs) and a common Y-shaped adapter (Integrated DNA Technologies) was ligated to all DNA libraries. Unique, asymmetric 10 base pair barcodes were added to the DNA fragment during library amplification with KAPA HiFi polymerase (KAPA Biosystems) to facilitate multiplexed exome capture and sequencing. Equal amounts of sample were pooled prior to overnight exome capture, approximately 16 hours, with a slightly modified version of IDT's xGen probe library; supplemental probes were added to capture regions of the genome well-covered by a previous capture reagent (NimbleGen VCRome) but poorly covered by the standard xGen probes. In total, n=38,997,831 bases were included in the targeted regions. Captured fragments were bound to streptavidin-coupled DYNABEADS®(Thermo Fisher Scientific) and non-specific DNA fragments removed through a series of stringent washes using the xGen Hybridization and Wash kit according to the manufacturer's recommended protocol (Integrated DNA Technologies). The captured DNA was PCR amplified with KAPA HiFi and quantified by qPCR with a KAPA Library Quantification Kit (KAPA Biosystems). The multiplexed samples were pooled and then sequenced using 75 base pair paired-end reads with two 10 base pair index reads on the Illumina NOVASEQ® 6000 platform using S2 flow cells.

### Sequence Alignment, Variant Identification, and Genotype Assignment

Upon completion of sequencing, raw data from each Illumina NOVASEQ® run was gathered in local buffer storage and uploaded to the DNAnexus platform for automated analysis. After upload was complete, analysis began with the conversion of CBCL files to FASTQ-formatted reads and assigned, via specific barcodes, to samples using the bcl2fastq conversion software (Illumina Inc., San Diego, CA). Sample-specific FASTQ files, representing all the reads generated for that sample, were then aligned to the GRCh38 genome reference with BWA-mem. The resultant binary alignment file (BAM) for each sample contained the mapped reads' genomic coordinates, quality information, and the degree to which a particular read differed from the reference at its mapped location. Aligned reads in the BAM file were then evaluated to identify and flag duplicate reads with the Picard MarkDuplicates tool (world wide web at “picard.sourceforge.net”), producing an alignment file (duplicatesMarked.BAM) with all potential duplicate reads marked for exclusion in downstream analyses.

GVCF files, including variant calls, were then produced on each individual sample using the WeCall variant caller (world wide web at “github.com/Genomicsplc/wecall”) from Genomics PLC, identifying both SNVs and INDELs as compared to the reference. Additionally, each GVCF file carried the zygosity of each variant, read counts of both reference and alternate alleles, genotype quality representing the confidence of the genotype call, and the overall quality of the variant call at that position.

Upon completion of variant calling, individual sample BAM files were converted to fully lossless CRAM files using samtools. Metric statistics were captured for each sample to evaluate capture, alignment, insert size, and variant calling quality, using Picard (world wide web at “picard.sourceforge.net”), bcftools (world wide web at “samtools.github.io/bcftools”), and FastQC (world wide web at “bioinformatics.babraham.ac.uk/projects/fastqc”).

Following completion of sample sequencing, samples showing disagreement between genetically-determined and reported sex (n=15), high rates of heterozygosity/contamination (D-stat>0.4) (n=7), low sequence coverage (less than 85% of targeted bases achieving 20× coverage) (n=1), or genetically-identified sample duplicates (n=14), and WES variants discordant with genotyping chip (n=9) were excluded. Six samples failed quality control in multiple categories, resulting in 38 individuals being excluded. The remaining 49,960 samples were then used to compile a project-level VCF (PVCF) for downstream analysis. The PVCF was created using the GLnexus joint genotyping tool. Care was taken to carry all homozygous reference, heterozygous, homozygous alternate, and no-call genotypes into the project-level VCF. An additional filtered PVCF, ‘Goldilocks’, was also generated. In the filtered Goldilocks PVCF, samples carrying SNP variant calls in the single sample pipeline or a DP<7 were converted to ‘No-Call’. After the application of the DP filter, sites where all remaining samples were called as Heterozygous and all samples have an AB<85% ref/15% alt were excluded. Samples carrying INDEL variant calls in the single sample pipeline with a DP<10 were converted to ‘No-Call’. After the application of the DP filter, sites where all remaining samples were called as Heterozygous and all samples have an AB<80% ref/20% alt were excluded. Multi-allelic variant sites in the PVCF file were normalized by left-alignment and represented as bi-allelic.

### Phenotype Definition

ICD10-based cases required one or more of the following: a primary diagnosis or a secondary diagnosis in in-patient Health Episode Statistics (HES) records. ICD10-based excludes had ≥1 primary or secondary diagnosis in the code range. ICD10-based controls were defined as those individuals that were not cases or excludes. Custom phenotype definitions included one or more of the following: ICD-10 diagnosis, self-reported illness from verbal interview and doctor-diagnosed illness from online-follow-up, touchscreen information. Quantitative traits (such as, physical measures, blood counts, cognitive function tests, and imaging derived phenotypes) were downloaded from UK Biobank (UKB) repository and spanned one or more visits. In total, 1,073 binary traits with case count ≥50 and 669 number of quantitative traits, were tested in WES association analyses.

### Annotation of Predicted Loss-of-Function (LOF) Variants

Variants were annotated using snpEff and gene models from Ensembl Release 85. A comprehensive and high quality transcript set was obtained for protein coding regions which included all protein coding transcripts with an annotated Start and Stop codon from the Ensembl gene models. Variants annotated as stop_gained, start_lost, splice_donor, splice_acceptor, stop_lost and frameshift are considered to be LOF variants.

A recent large-scale study of genetic variation in 141,456 individuals provides a catalog of LOF variants. A direct comparison to this data is difficult due to numerous factors such as differences in exome sequencing capture platforms, variant calling algorithms and annotation. Additionally, the number of individuals and the geographic distribution of ascertainment (and thus genetic diversity) in the NFE subset of gnomAD may be larger than that of UK Biobank with WES in this report. Nonetheless, the gnomAD exome sites labeled as “PASS” from gnomAD r2.1 were annotated using the annotation pipeline. Data from gnomAD were lifted over to HG38 using Picard LiftoverVcf. The data was subset to Non-Finnish Europeans (NFE) (n=56,885 samples), individuals) restricted to variants with MAF_{NFE}<1% and obtained 261,309 LOFs in any transcript in 17,951 genes. Restricting LOFs only to those that are present in all transcripts, 175,162 LOFs were observed in 16,462 genes. 134,745 LOFs were observed in all transcripts of genes in UKB participants with WES of European ancestry.

### Methods for LOF Burden Association Analysis

Burden tests of association were performed for rare LOFs within 49,960 individuals of European ancestry. For each gene region as defined by Ensembl. LOFs with MAF≤0.01 were collapsed such that any individual that is heterozygous for at least one LOF in that gene region is considered heterozygous, and only individuals that carry two copies of the same LOF are considered homozygous. Rare variants were not phased, and so compound heterozygotes are not considered in this analysis.

For each gene region, 668 rank-based inverse normal transformed (RINT) quantitative measures (including all subjects and sex-stratified models) with ≥5 individuals with non-missing phenotype information were assessed using an additive mixed model implemented in BOLT-LMM v2. Prior to normalization, traits were first transformed as appropriate (log 10, square) and adjusted for a standard set of covariates including age, sex, study site, first four principal components of ancestry, and in some cases BMI and/or smoking status. Data-points greater than five median absolute deviations from the median were excluded as outliers prior to normalization. 1,073 discrete outcomes (including all subjects and sex-stratified models) with ≥50 cases were assessed with covariate adjustment for age, sex and first four principle components of ancestry using a generalized mixed model implemented in SAIGE. For each quantitative and discrete trait included in the analysis, only gene regions in which >3 LOF carriers with non-missing phenotype and covariate information were evaluated.

Positive controls were systematically defined using a two-step approach. First, each gene for relevant disease, trait, biological, or functional evidence was annotated using publicly available resources including OMIM, NCBI MedGen, and the NHGRI-EBI GWAS catalogue. Genes with supporting evidence from at least one source, were then manually curated using NCBI PubMed to verify the relationship between the trait and LOF variants in the gene of interest. Genes with locus-level support for the trait of interest or related phenotype(s) in the GWAS catalog but lacking clear supporting evidence for a LOF association are reported herein as novel LOF associations.

### Methods for Single Variant LOF Association Analysis

Single variant association analysis was performed using the same methods as described in the methods section for burden association analysis. For gene-trait associations with p<10⁻⁷, single variant association statistics was calculated with the phenotype of interest for all LOFs included in the burden test that are observed with a minor allele count ≥5 in the 49,960 European ancestry individuals with WES. Association statistics for these variants are reported in Extended Data (ExtData_SingleVariantLOFs_V1.xlsx).

P-VAL Leave-One-Out (LOO) Burden is the p-value of the absence/presence test excluding the variant being tested. Delta P-Val Burden is the ratio of the p-value in the drop-one-out analysis compared to the burden test using all 65 variants. Burden summary statistics using all 65 variants in unrelated individuals: B=1.44, SE=0.26, p-value=3.12E-08, cMAF=0.00174, cMAC=152, cMAF_cases=0.0066, cMAC_cases=17, cMAF_controls=0.0016, cMAC_cases=135. Stepwise logistic regression selected 11/65 variants (AIC 11451). Burden summary statistics using the 11 variants: B=3.71, SE=0.437, p-value<2e-16, cMAF=0.0003, cMAC=22, cMAF_cases=0.005, cMAC_cases=12, cMAF_controls=0.0001, cMAC_controls=10. When analysis is restricted to variants with MAC>1, stepwise logistic regression selects 5/13 variants (AIC 11484). Burden summary statistics using the 5 variants: B=3.02, SE=0.53, p-value=8.92e-09.

LD (r2) across the 11 pLOF variants selected by stepwise regression for PIEZO1 and a positive control (16: 88835545_G_A; highlighted in green) reported in the literature for PIEZO1. None of these 12 variants are in LD, R2>0.01. When the burden test is adjusted for the previously reported variant rs2911463 (16: 88835545_G_A), the burden test p-value remains <2E-16 (AIC 11,444), which indicates that the burden is not tagging the reported variant.

### Example 2: Demographics and Clinical Characteristics of Sequenced Participants

A total of 50,000 participants were selected, prioritizing individuals with more complete phenotype data: those with whole body MRI imaging data from the UK Biobank Imaging Study, enhanced baseline measurements, hospital episode statistics (HES), and/or linked primary care records (which will soon be available to approved researchers). During data generation, samples from 40 participants were excluded due to failed quality control measures or participant withdrawal, resulting in a final set of 49,960 individuals. Overall, the sequenced sample is representative of the 500,000 UKB participants (Table 1). There were no notable differences in age, sex, or ancestry between the sequenced sample and overall study population. Sequenced participants were more likely to have HES diagnosis codes (84.2% among sequenced vs. 77.3% overall) and enhanced measures (Table 1).

TABLE 1
Clinical characteristics in whole exome sequenced
and all UK Biobank participants

| Basic Demographics and Clinical | UKB 50k WES | | UKB 500k | |
|---|---|---|---|---|
| Characteristics | Participants | | Participants | |
| N | 49,960 | | 502,543 | |
| Female, n(%) | 27,243 | (54) | 273,460 | (54) |
| Age at assessment, years | 58 | (45-71) | 58 | (45-71) |
| Body mass index, kg/m² | 26 | (21-31) | 26 | (21-31) |
| Number of imaged participants | 12,075^{b} | | 21,407ª | |
| Number of current/past smokers, n(%) | 17,515 | (35) | 216,482 | (43) |
| Townsend Deprivation Index | −2.0 | (−6.1, −2.1) | −2.13 | (−6.2, −1.97) |
| Inpatient ICD10 codes per patient | 5 | | 5 | |
| Patients with >=1 ICD10 diagnoses (%) | 84.2 | | 78.0 | |
| Genetic Ancestry Assignment^{c} | | | | |
| African (%) | 1.49 | | 1.24 | |
| East Asian (%) | 0.54 | | 0.51 | |
| European (%) | 93.66 | | 94.55 | |
| Cardiometabolic phenotypes | | | | |
| Coronary Disease, n(%) | 3,3340 | (6.6) | 35,879 | (7.1) |
| Heart Failure, n(%) | 300 | (0.6) | 4,399 | (0.8) |
| Type 2 Diabetes, n(%) | 1,541 | (3.0) | 17,261 | (3.4) |
| Respiratory and immunological | | | | |
| phenotypes | | | | |
| Asthma, n(%) | 8,250 | (16) | 68,149 | (13) |
| COPD, n(%) | 741 | (1.4) | 7,438 | (1.4) |
| Rheumatoid Arthritis, n(%) | 710 | (1.4) | 7,337 | (1.4) |
| Inflammatory Bowel Disease n(%) | 543 | (1.0) | 5,783 | (1.1) |
| Neurodegenerative phenotypes | | | | |
| Alzheimer's Disease, n(%) | 13 | (0.05) | 320 | (0.06) |
| Parkinson's Disease, n(%) | 65 | (0.13) | 1,043 | (0.2) |
| Multiple Sclerosis, n(%) | 126 | (0.25) | 1,352 | (0.26) |
| Myasthenia Gravis, n(%) | 14 | (0.02) | 217 | (0.04) |
| Oncology phenotypes | | | | |
| Breast Cancer, n(%) | 1,667 | (3.3) | 16,887 | (3.3) |
| Ovarian Cancer, n(%) | 162 | (0.3) | 1,777 | (0.3) |
| Pancreatic Cancer, n(%) | 602 | (1.2) | 4,611 | (0.9) |
| Prostate Cancer, n(%) | 848 | (1.6) | 8,855 | (1.7) |
| Melanoma, n(%) | 598 | (1.1) | 5,715 | (1.1) |
| Enhanced measures | | | | |
| Hearing test available, n(%) | 40,546 | (81.1) | 167,011 | (33.2) |
| Pulse Rate, n(%) | 40,548 | (34.2) | 170,761 | (33.9) |
| Visual Acuity Measured, n(%) | 39,461 | (78.9) | 117,092 | (23.2) |
| IOP measured (left), n(%) | 37,940 | (75.9) | 111,942 | (22.2) |
| Autorefraction, n(%) | 36,067 | (72.1) | 105,989 | (21.0) |
| Retinal OCT, n(%) | 32,748 | (65.5) | 67,708 | (13.4) |
| ECG at rest, n(%) | 10,829 | (27.1) | 13,572 | (2.1) |
| Cognitive Function, n(%) | 9,511 | (19.0) | 96,362 | (19.1) |
| Digestive Health, n(%) | 13,553 | (28.1) | 142,310 | (28.3) |
| Physical Activity Measurement, n(%) | 10,684 | (21.3) | 101,117 | (20.1) |

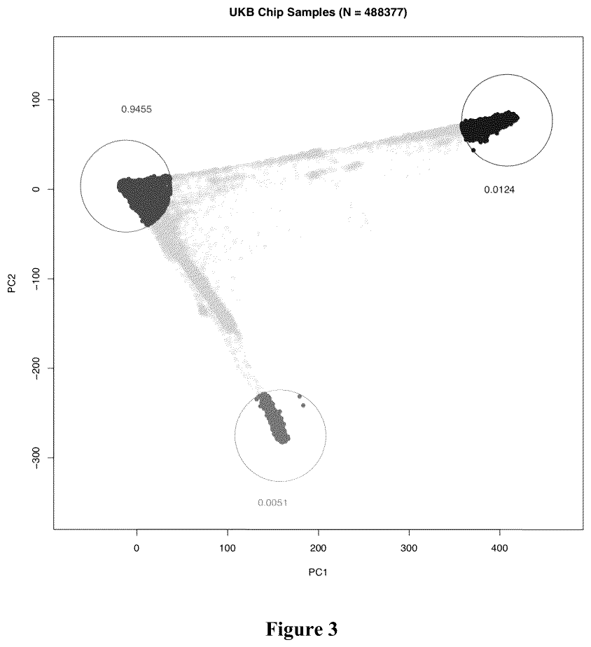

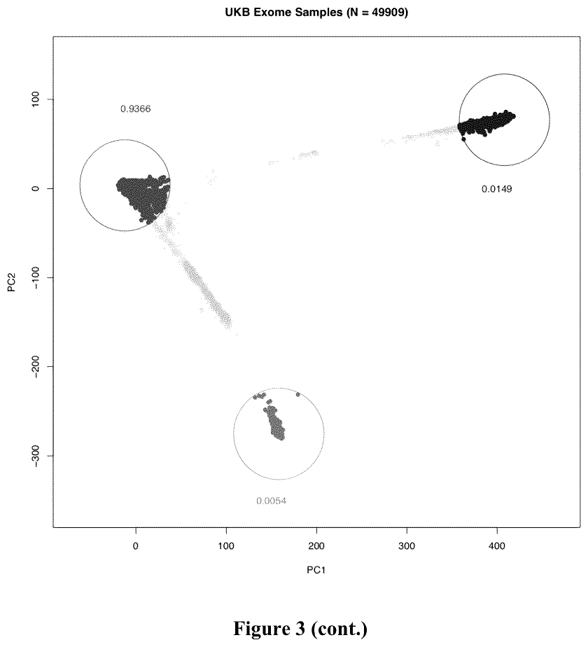

^{a}The number of samples with at least one non-missing image derived phenotype value from data downloaded from UK Biobank in November 2018.
^{b}The number of samples with exome sequencing data and at least one non-missing image derived phenotype value from data downloaded from UK Biobank in November 2018.
^{c}Number of samples in 3 pre-defined regions of a plot of the first two genetic principal component scores, where the regions are selected to represent African, East Asian, and European ancestry (see, FIG. 3).

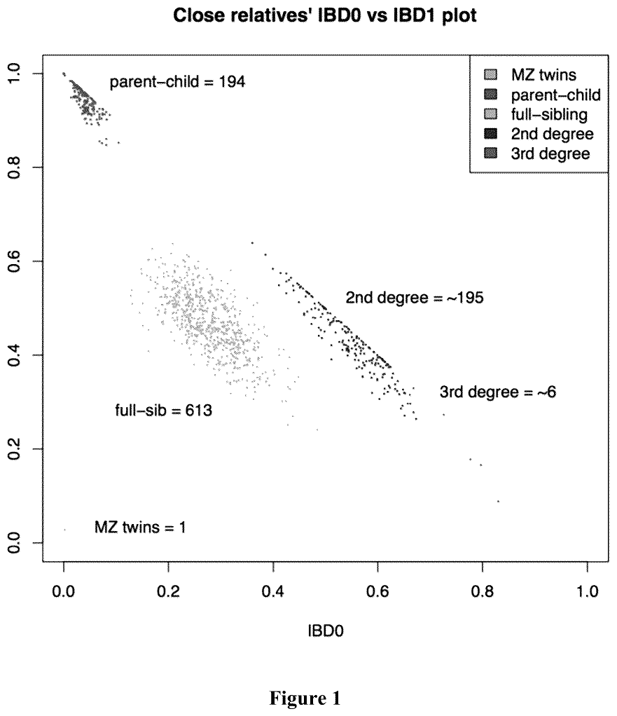

Participants with WES with at least one HES diagnosis code did not differ from non-sequenced participants in the median number of primary and secondary ICD10 codes or broad phenotype distributions, other than codes for asthma (ICD10 J45) and status asthmaticus (ICD10 J46), as the most enriched in sequenced samples, and senile cataract (ICD10 H25) and unknown and unspecified causes of morbidity (ICD10 R69), as the most depleted. The sequenced subset includes 194 parent-offspring pairs, 613 full-sibling pairs, 1 monozygotic twin pairs and 195 second degree relationships. The distribution of relatedness between pairs of individuals in UKB WES are included in FIG. 1.

### Example 3: Summary and Characterization of Coding Variation from WES

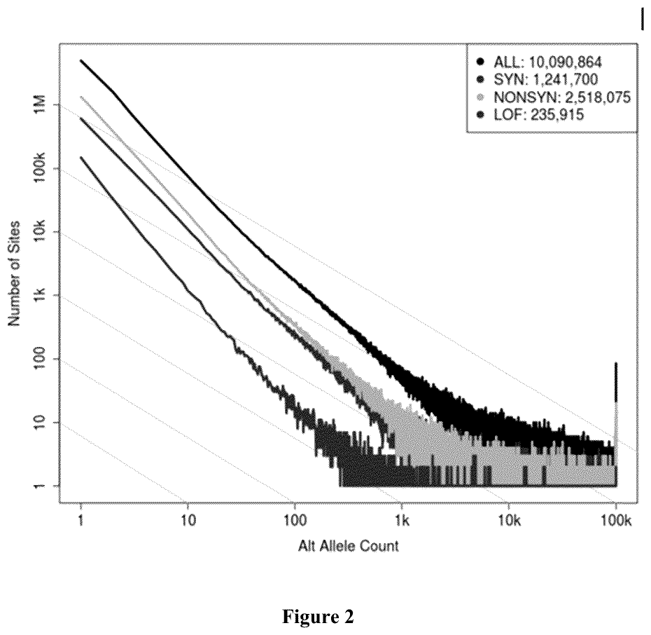

The protein coding regions and exon-intron splice sites of 19,467 genes were targeted. Counts of autosomal variants observed across all individuals by type/functional class for all and for MAF<1% frequency. All variants passed QC criteria, individual and variant missingness <10%, and Hardy Weinberg p-value>10⁻¹⁵. Median count of variants and interquartile range (IQR) for all variants and for MAF<1%. The average proportion of targeted bases (n=38,997,831) achieving at least 20× coverage in each sample was 94.6% (standard deviation 2.1%). 10,028,025 single nucleotide and indel variants were observed after quality control, 98.5% with minor allele frequency (MAF)<1% (Table 2). Of the total variants, 3,995,785 are within targeted regions. These variants included 2,431,680 non-synonymous (98.9% with MAF<1%), 1,200,882 synonymous (97.8% with MAF<1%), and 205,867 predicted loss of function (pLOF) variants affecting at least one coding transcript (initiation codon loss, premature stop codons, splicing, and frameshifting indel variants; 99.7% with MAF<1%) (FIG. 2). The tally of 9,403 synonymous (IQR 125), 8,369 non-synonymous (IQR 132) and 161 pLOF variants (IQR 14) per individual (median values) is comparable to previous exome sequencing studies. If the analysis is restricted to pLOF variants that affect all transcripts for a gene, the number of pLOF variants drops to 140,850 overall and 96 per individual (a reduction of about 31.6% and about 40.4%, respectively), consistent with previous studies.

TABLE 2
Summary statistics for variants in sequenced exomes of 49,960 UKB participants

| | WES in n = 49,960 autosomes | | Median Per Participant (IQR) | | | |
|---|---|---|---|---|---|---|
| | | # Variants | | | # Variants | |
| | # Variants | MAF < 1% | # Variants | | MAF < 1% | |
| Total | 10,028,025 | 9,882,400 | 49,000 | (628) | 1,626 | (133) |
| Targeted Regions | 3,995,785 | 3,941,162 | 18,670 | (235) | 640 | (56) |
| Variant Type | | | | | | |
| SNVs | 3,823,276 | 3,770,454 | 18,404 | (233) | 613 | (54) |
| Indels | 142,603 | 141,439 | 266 | (16) | 21 | (25) |
| Multi-Allelic | 466,433 | 459,434 | 2,304 | (50) | 84 | (15) |
| Functional Prediction | | | | | | |
| Synonymous | 1,200,882 | 1,175,279 | 9,403 | (125) | 222 | (26) |
| Missense | 2,431,680 | 2,406,367 | 8,369 | (132) | 367 | (38) |
| pLOF (any transcript) | 205,867 | 205,215 | 161 | (14) | 20 | (7) |
| pLOF (all transcripts) | 140,850 | 140,445 | 96 | (10) | 14 | (6) |

### Example 4: Phenotypic Associations with LOF Variation

The combination of WES and rich health information allows for broad investigation of the phenotypic consequences of human genetic variation. LOF variation can yield tremendous insights into gene function; however, imputed datasets are missing the majority of such variation. WES is well-suited to identify LOF variants and to evaluate their phenotypic associations. Gene burden tests of associations for rare (AAF<1%) pLOF variants (pLOF variants identified in WES across all genes with >3 pLOF variant carriers) were conducted with 1,741 traits (1,073 discrete traits with at least 50 case counts defined by hospital episode statistics and self-report data, 668 quantitative, anthropometric, and blood traits) in n=46,979 individuals of primarily European ancestry. For each gene-trait association, the strength of association for the pLOF gene burden test was also compared to the association results for each of the SNVs included in the burden test.

### Example 5: LOF Associations and Novel Gene Discovery

In the pLOF gene burden association analysis, a novel association between PIEZO1 LOFs (cumulative allele frequency=0.2%) and greatly increased risk for varicose veins was identified. Results for PIEZO1 for the binary phenotype of asymptomatic varicose veins of lower extremities within the UKB 50 k exome and UKB 150 k exome are shown in Table 3.

TABLE 3
PIEZO1 LOF gene burden associations

| | Counts | OR | | N | Lowest P |
|---|---|---|---|---|---|
| Exome | RR\| RA\|AA | (95% Cl) | Burden P | SNV | SNV |
| UKB | Ctrls: 43285\|142\|0 | 4.9 | 2.7 × 10⁻⁸ | 65 | 2.29E−3 |
| 50k | Cases: 1267\|20\|0 | (3.1, 7.8) | | | |
| UKB | Ctrls: 131514\|443\|0 | 3.0 | 1.8 × 10⁻⁸ | | |
| 150k | Cases: 3559\|36\|0 | (2.1, 4.4) | | | |

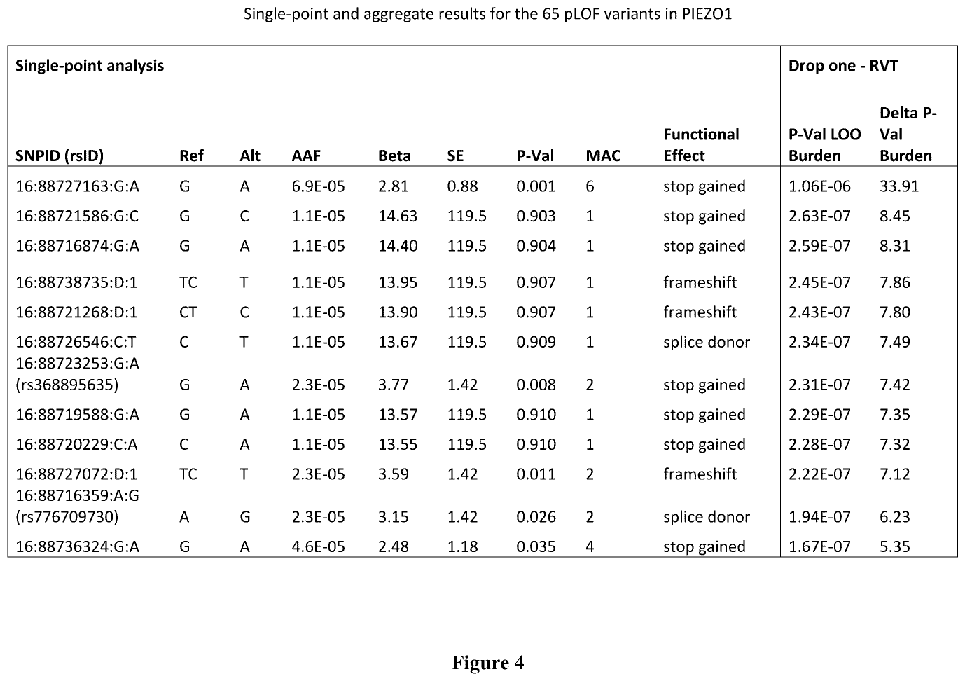

This finding is driven by a burden of rare LOF variants, with the most significant PIEZO1 single variant LOF association in the UKB 50 k exome achieving a p-value of 2.29×10⁻³. Leave one out analyses of the UKB 50 k exome indicate no single variant accounts for the entire signal and step-wise regression analyses indicated that 11 separate variants (5 of which had MAC>1) were contributing to the overall burden signal (FIG. 4).

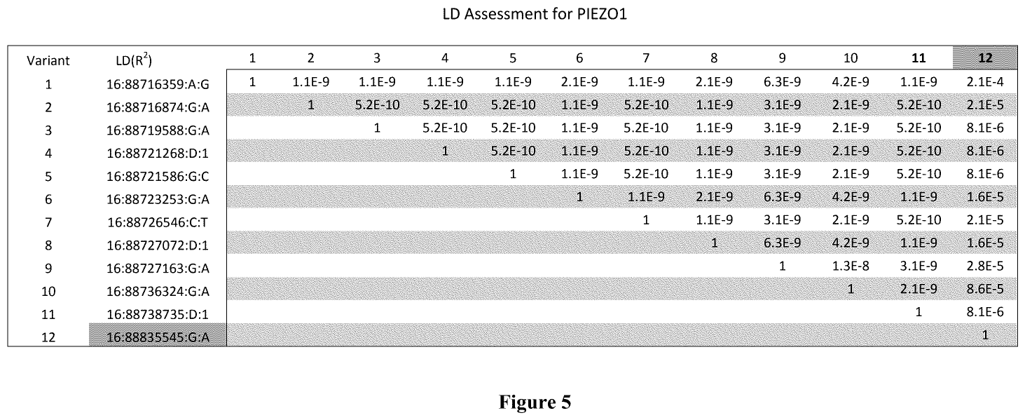

This finding was replicated in 2,953 varicose veins cases and 75,694 controls previously exome sequenced (OR=2.7, p=1.86×10⁻⁹). This region had previously been implicated by common non-coding variants with small effects on disease risk, where rs2911463 and other nearby common variants on chromosome 16 have recently been associated with varicose veins (frequency=0.69, OR=0.996, p-value=1.0×10⁻²⁷ in GWAS of 408,455 genotyped U.K. Biobank participants). The rs2911463 variant is not in LD with any of the key variants identified (FIG. 5) and the burden test remains significant when adjusting for rs2911463.

1. A method of identifying a human subject having an increased risk of developing varicose veins, wherein the method comprises determining or having determined in a biological sample obtained from the subject the presence or absence of:
a Piezo Type Mechanosensitive Ion Channel Component 1 (PIEZO1) predicted loss-of-function variant genomic nucleic acid molecule;
a PIEZO1 predicted loss-of-function variant mRNA molecule;
a PIEZO1 predicted loss-of-function variant cDNA molecule produced from the mRNA molecule; or
a PIEZO1 predicted loss-of-function variant polypeptide;
wherein:
the absence of the PIEZO1 predicted loss-of-function variant genomic nucleic acid molecule, mRNA molecule, cDNA molecule, or polypeptide indicates that the subject does not have an increased risk for developing varicose veins; and
the presence of the PIEZO1 predicted loss-of-function variant genomic nucleic acid molecule, mRNA molecule, cDNA molecule, or polypeptide indicates that the subject has an increased risk for developing varicose veins.

2. A method of diagnosing varicose veins in a human subject, wherein the method comprises detecting in a sample obtained from the subject the presence or absence of:
a Piezo Type Mechanosensitive Ion Channel Component 1 (PIEZO1) predicted loss-of-function variant genomic nucleic acid molecule;
a PIEZO1 predicted loss-of-function variant mRNA molecule;
a PIEZO1 predicted loss-of-function variant cDNA molecule produced from the mRNA molecule; or
a PIEZO1 predicted loss-of-function variant polypeptide;
wherein when the subject has a PIEZO1 predicted loss-of-function variant genomic nucleic acid molecule, mRNA molecule, cDNA molecule, or polypeptide, and has one or more symptoms of varicose veins, then the subject is diagnosed as having varicose veins.

3. (canceled).

4. A method of treating a patient with a therapeutic agent that treats or inhibits varicose veins, wherein the patient is suffering from varicose veins or has an increased risk of developing varicose veins, the method comprising the steps of:
determining whether the patient has a Piezo Type Mechanosensitive Ion Channel Component 1 (PIEZO1) predicted loss-of-function variant nucleic acid molecule encoding a human PIEZO1 polypeptide by:
obtaining or having obtained a biological sample from the patient; and
performing or having performed a genotyping assay on the biological sample to determine if the patient has a genotype comprising the PIEZO1 predicted loss-of-function variant nucleic acid molecule; and
when the patient is PIEZO1 reference, then administering or continuing to administer to the patient the therapeutic agent that treats or inhibits the varicose veins in a standard dosage amount; and
when the patient is heterozygous or homozygous for a PIEZO1 predicted loss-of-function variant nucleic acid molecule, then administering or continuing to administer to the patient the therapeutic agent that treats or inhibits the varicose veins in an amount that is the same as or greater than the standard dosage amount;
wherein the presence of a genotype having the PIEZO1 predicted loss-of-function variant nucleic acid molecule encoding the human PIEZO1 polypeptide indicates the patient has an increased risk of developing varicose veins.

5. The method according to claim 4, wherein the determining step is carried out in vitro.

6. The method according to claim 5, wherein the determining step comprises sequencing at least a portion of the nucleotide sequence of the PIEZO1 nucleic acid molecule in the biological sample, wherein the sequenced portion comprises a position corresponding to a predicted loss-of-function variant position, wherein when a variant nucleotide at the predicted loss-of-function variant position is detected, the PIEZO1 nucleic acid molecule in the biological sample is a PIEZO1 predicted loss-of-function variant nucleic acid molecule.

7. The method according to claim 4, wherein the determining step comprises:
a) contacting the biological sample with a primer hybridizing to a portion of the nucleotide sequence of the PIEZO1 nucleic acid molecule that is proximate to a predicted loss-of-function variant position;
b) extending the primer at least through the predicted loss-of-function variant position; and
c) determining whether the extension product of the primer comprises a variant nucleotide at the predicted loss-of-function variant position.

8. The method according to claim 6, wherein the determining step comprises sequencing the entire nucleic acid molecule.

9. The method according to claim 4, wherein the determining step comprises:
a) amplifying at least a portion of the PIEZO1 nucleic acid molecule that encodes the human PIEZO1 polypeptide, wherein the portion comprises a predicted loss-of-function variant position;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the predicted loss-of-function variant position; and
d) detecting the detectable label.

10. The method according to claim 9, wherein the nucleic acid molecule in the sample is mRNA and the mRNA is reverse-transcribed into a cDNA prior to the amplifying step.

11. The method according to claim 9, wherein the determining step comprises:
contacting the nucleic acid molecule in the biological sample with an alteration-specific probe comprising a detectable label, wherein the alteration-specific probe comprises a nucleotide sequence which hybridizes under stringent conditions to a predicted loss-of-function variant position; and
detecting the detectable label.

12. The method according to claim 4, wherein the PIEZO1 predicted loss-of-function variant nucleic acid molecule is 16:88715629:G:A, 16:88715728:G:T, 16:88715767:G:A, 16:88715802:C:A, 16:88715822:D:4, 16:88715987:I:1, 16:88716359:A:G, 16:88716570:C:T, 16:88716874:G:A, 16:88717213:T:A, 16:88719588:G:A, 16:88719722:C:G, 16:88719870:G:T, 16:88720068:D:2, 16:88720229:C:A, 16:88720248:D:4, 16:88720394:C:T, 16:88720644:D:1, 16:88720698:D:1, 16:88720698:I:1, 16:88721165:C:A, 16:88721268:D:1, 16:88721307:G:A, 16:88721586:G:C, 16:88721652:G:C, 16:88722217:C:T, 16:88722605:I:1, 16:88723005:I:7, 16:88723253:G:A, 16:88723311:C:T, 16:88725081:C:A, 16:88726282:G:A, 16:88726546:C:T, 16:88726619:G:A, 16:88726924:G:A, 16:88727038:C:T, 16:88727072:D:1, 16:88727163:G:A, 16:88731768:D:1, 16:88732334: C:G, 16:88732411:D:1, 16:88732720:D:1, 16:88733326:G:C, 16:88733337:D:4, 16:88733587:C:A, 16:88733965:D:1, 16:88734017:C:A, 16:88734042:I:1, 16:88734679:C:T, 16:88734909:I:1, 16:88736167:D:2, 16:88736324:G:A, 16:88736391:G:T, 16:88736409:C:T, 16:88736671:G:A, 16:88737557:A:C, 16:88737727:C:G, 16:88737815:C:T, 16:88738283:G:C, 16:88738637:G:A, 16:88738735:D:1, 16:88741477:C:T, 16:88742306:D:1, 16:88749399:G:A, or 16:88784929:C:T, or an mRNA molecule produced therefrom, or a cDNA molecule produced from the mRNA molecule.


SEQUENCE LISTING (4 sequences)

SEQ ID NO: 1 - DNA, 69883 bp, Homo sapiens
  source 1..69883: mol_type = genomic DNA
gggagccgccgtccggcccagctcggccccagtgagccgagcgctgcgctccgccgaggggcagggcggtcgcctgagcgagcgcgggcccgggacgtcggcaccggcggggcggccgaaggagaaggaggaagaggagaaggcggcgcgcgggtccccgcgggtcagccatggcgcgccggccccggggcccccgcaccgccccatagcgccgcggcgtccgctcggtctgggccgggccctgggccctccagccatggagccgcacgtgctcggcgcggtcctgtactggctgctgctgccctgcgcgctgctggctggtgagtggggggcgggcgcctgggggcgacgggagggggctgcgtctcggctccccacggcctggacaccggacgacgccggccggggcgagggctgcgggcgagcgggcgcggaaattcccagggacgcgcgacccgggcgcccgcattcctgaagcatgagcgcgccaggcggcggcggggctcctgtcccagggccgggctggaagggcggcggcggctgggggagacggcaccgcgtgcccacgggggcggtcgagcgagcgccgggcatagcgcggctggcgtctccgccggggcgctgcggagaggaggccgccgggcgaggcggtgtttgccccggtgggaagggccgcggcggtggtggggggagcacgaatctctttttctctttcgggtttaaaaaaaaaagcgcaaagttgcatcaggacttcctgacaatctgggagaaggcgggcttcctgcctggagctgtttaatttggagcttcccgagcccaacgaacgtccgtgcccagggcccagccccgctcaccgctgcacccccctctgccggactgaggcggtcccacactttggaaaaaaatagtgtgggttcctccctggtcctcccttgccctactgggctcagtttcgcaggggcgggggccggcctctgccctggtctgggggaggggacacccccggaggctgtggcctggtgtcagggcggggcaggggtccccagtcctggcatctgtgttccctgcttgccgggcagtggtgcccctttcgcgaagcacacccgggtggcttggtgctgcacggcctggcacccctacccttccccgaccctggcctagccgggacccagggtccgcgccctccgcccgggggctccccacgtgtgattgatctgggaagcagtcggatggaattaacccacggacaagtgggacggtttgcattgggagtccgccatggacacggcaggtggggccttttgattgtaaaagccctttcggagcccttgcctcgctccaggtgggagctcgcccagcgctagctttggggatctagagccgcctgcctgaggctcccagacagactgcgttttgatcggtcgcacagaaaggtggtgaaacttggggaagattttctagacaggaatcaatgaaaaccattgaggctggagaggagaggttttgagcaactctcttcagtgcggtcagccctgtgtggactgggcagcctgggacctgctcccagtgcagggtcagatgggccgtaaacagggcccggctgtgttccttcctgtgccttgaaaacaagcaggacagcctggcacagaggcagagtctagagctgacaggccttagagagggaaacaggaaagcttctgaaacgtcccgttcacactgatcgttccatttcctcttgtgtctgagtgggagcgggtgtcctccctgcagggaatgccccccctctcagatggcagctgctccttgggcagagttggcaatgtttttctttaaatgaccagatggtaaatattttcatgtcacaaaatcttcttcttctggtatttttccagccattaaatgtaaaagccctcctgagttcatgggctgtacctaaacaggtgttgagcccgattctgtggcacatgtggtttgctgccccctgggcattggtcagggggcctgggttctgccttctcgattgctatccgcgtgggggatctgggggagggatcactgttcttcttgcttttggcctccttggggaggatggggaggtagcccaggggtgctcacccaggccccgtgtcagtcttctatgaaacttttaaagaatagtgatgactgactgtctgtctgtatggtactttccttaaacctaaaactggtcccaaataaagtctcttaatttgaaagatgctgaagcccgggccataccccacactgattctgtgtctggggatggggcgtggggcctgggcctcactcagtgttttctctcagtcacctgggggagatggaagtggagccggccaagaaccctgcctgcctgcctgctggccgggactcctgagtcaggctctctggccctggggtgtgggcagctccagatggacccgcgatgtgcaggttcagctggcctggccggaggtgggacactggctttgctgtctttggagtgccccctccctctctggcgagctttggctggaagcagttctaccgtgttttggaaatgaatgaggccttcagaaggcattagtcagtgtgtgcctgcgctggctcagacagtgcctggtgagggtttgagtcatcctggggtgcccctggcccccacgccctccctctccagtgcaggatcattacccaaaaatctggcagggagctgccccacccacagggagcaggggcctccttcagcagtctcacctaatgttgctggagccttgggggatcagggcccatctcttctagagagatgtcagggcagggctgggcgcgatggctcacacctgtaatcccagagcttcgggaggccaaggtgggaggattgcttgagcgtagccattcgagagcagcctgggcaacgtagagatccccatctctatgaaaaatatttaaaaattagctgggcatggtggtagtgcacctgtagtcccagctactcaggaggccaaggtgggatgattgtttgagtccaggagttggaggctacagtgagccatgattgtatgactgcactccagcctgggtgacagaccctgtctcaaaaaagaaaaaggaaaaaaaagggcaggacatttgtgattctgataatatgggaccgcacctccagttctatcagtgggaaatctagacagggctgcggaaagccagctggtgcgagaggagccaccgtgtcactgactgtgggacacccacgtgggctgacaatatggcttctgcttttcagggtgcctcgtggcacagcctaggggcacacccacggccggcaagctgggcgtcacctccttcaggctgattgtcactgagaagtgtcaccatttagcatgagggatgctgcctcctttttcagaacattgtcaccatcagggtctcaccactcctgggaggcggccgagaagctggggaacagcaggcactcggctcacagttgctcagcagtgcagaccctctgagctgagcatggcagagtcacccttcggaggcctgtgcccgggtctcaggacctgcacagaaccctggcctgtcccatccgagggtgctgggaagagcatggccgtggcagagtagggtgggagctgctttcctctgtggcttgggggccccttctgagcatcagctccctggtgtgacagaggggcgcactctgtccccatgctgggcctggaggctggatgagtcagcaggagagcctggggcctgcctcacagcaccaagggctgcaggtgtgagtgtgcacatgtgtgcgtgtttggggaaggggccagggactgcccaggagctgaggatgggtcacagcgggtgctcgtcccgcagcgggtcactggtgcccaggacacagggagctccagccccagctgccagggtcccacagagaggaagtttcctctgggggtgggtgggggcgcacagtctctgatcctggccccaaggcagcttcctgggcggtgtctctcctgtgctgactcggcagtgcatttgctttcggtgctcaaagatgaaggggaaccaccgtgggccttgacggcctcatctgcccgctgcagcccactcctgagatgggaccaccgcagtcgtcagggtccagtgagagccgcatcttgcaggaagccattcctggcctctctggcctcagaaatccccttttcaattcaacaaaatgttagtcttctgtttagctattttagaaatagacagttaggcttttttctcttttttcttaaagacagaggctcactctgtcgcccaggctggagtgtagcggcgtgattttggctccctacaacttcagcctccagggctcaagccatcctcctgcctcagcctcctgagtagctgagattacaggtgtgtagcaccatacctaatttttgtattttttgtagagacggggtttcaccactttggccaggctggccttgaactcctgaccttaagcgatctgcccgcctcagcctccaaaagtgctggggttacaggcatgagccaccgcgcccggctgacagttagacttttgcttctttgtttatataagctttttcttctggttccaaaagcaagtttgcccttccttgtggtagagaatcctagctcacagagcagtttagaagccagcacagtatcccacacacacatctggcatggacagacccttccttgctgggtgtgggtcctgtgttcttctgaaaggcaagctgttcccagccaacccctgcccctctctgccttagcctgcctggaggcctgagtctcctgggtgactgtgaggtgggacccccccttcccctacccccacccacatcctctgtatctgccttctgtcctgctcttgacctttgagctcctctgctggcttcagggtgcggctgttgagcctattttttggattaggactctggggtgagggaagttaattcacacacccaagatcacactggtgggaagggacaggcccggggtgaaggcttctcctctccttggcggttgagtcccacacctgctggccgaggcacctgaaggggacttggggtccagggtcactgggaggacgggggcaggcagaggggtggccgacctggtggcggctcgtgggcagcagccgaccctatcttgctctgaacgtgtggggccctcacccccttctctgggtctgggtttcctcccctgtaagtgacaccgtaaaagcttcacagccgcttccaagtctcaggtctctcgggctttggatctcacacccaggctgggtgggggtaggggcgggacagccgtcccccccggaaggctcagaattcctcgcacaatcgtggggccaggagacccgcaacacaggctttcccagctgcgctgagtgccgcggtggccgggggtccgtcgggcctccatggagctgaggggaaggggccactcaccgcctggtcccggagcacacagggcagctcccaggagcacagaggctccttgggacctgtggggctgtcggcctccctcatgctgcacacacagcgcgtccccaggggtgtctgcagcccagcccatcccactgcagattcccgaccactcagattcattcatgcatcctctcacgccggtcctgtgggacagagctctgggcagcagccagaagtccaagttctggttcagggccagtggaggtgggtgttggggtggggctggagctccctgctctccctcccaagctagccaggaaaggaggttgggggccccgcacggtcattgctgtttattcacaaagcgcgatgctgagcacaggcggggaaagaaaagtgcgatcagtgccaggaaaatggggctcccccgacgccgtccaaaatgggatcccttgccgggcgcggtggctcacacctgtaatcccagcactttgggaagccgaggcaggtggatcacctgaggtcaggagttcgagaccagcctgaccaacatggcaaaactccatctcagctaaaaatacaaaaattagctgggcatggtggcgggcacctgtattcccagctgggaaggctgagacaggagaatcgccggaacccaggaggtagaggttgcagtgagccacagtcgtgccactgcactccagcttgggggcttgggggacagcaagactccctctcaaaaaaaaaaaaaaaaagtgaagtcgtaaatcagattaaattccctttttaaccctttgaacctctgtcctcccctgttcccagcgggaagcctctggtgaacgcgccatgcaccccacctgcccccgctctctgggtctctctcccagctggaacggccgcttccccaggtgccttccctgggccacagccttgtgcctcggcggctgctggatgcctgggtgtgtgggtggctcccagtgtgtgggatggcacacgagcctctcgcccttctgtgtggggtcgcacacccaccgcaggccgtatttttgctcacgttcatgtttctccacggtggacgctgggttgcagggacccttcctgtgtgcgggtgaggatctgggcagctgctggtgccgggcccatggggacgctgaccgtcccgggtgccggctctgaggtgtgcagtggacggctgtcctgccgggcgctgccagggccccttaggccgacgtgcgtggccacccgattccccgccgttgtctcaggaacctttgccgagtggggtggatcaatttttcgggtgtgttttaatagcataattacagggagtatttcaggctccctctgacgggccggcagggtttggctgccggctgtttaccaggctccaatctgcacactatttttctgtgggtatatatagctggggctgcttctccttcctcaggttcaggctaaagagggacagcagccgcctcagccaccccctgtggtttcctttgcctgtggatgggcggctaaaatgggcccaggaagagtcaagaacaaggccggctctcggtgccacagctctacccccaaaagcaggaagggggctcgggccatgcccatctgtgagctacaccggtccgggagcggcatcaggcaggggagtcctggacccccgcagtgctggggtgtgtttgtccgccctccctcccgtgtgtctagaagcctccagcctcggggaaaacaatgaaactcaactgtgacttaaacagattcccaggcccgcaggagctcccggaggcttgtggctgtggcgagacctggagggccatgcgggagggacagacgcaggtttgcggaggccgcctgcccaggaggggcgtcaaaggaggggacagatgtgggtttagggaggccacctgcccgggaggagcctcgaaggaaggcacaggcgtgggtttggggctgcctgccctggaggggcctcaaggaccccaggtctggtcttggtctcactcacctcctggaccccccaaggcctgcagtttgcaatctgtcgcctgggacccccactgtctgcctttacgcagctcagccaccacgcggccctcgctccctcatttacttgatttctgtttatggttaaagtaccgtttaaaacgacacatcattaaagcaacatgaaagggagttttgaaaagggaagccatcgtccatcccactgcccctgcctcagcggggatttacttttcctttcctgtctgcgggccagtgacaatgaggaccccgcaatgtgtctgcgggccagtgacaatgaggaccccgcaatgtgtctgcgggccagtgacagtgaggaccccgcaatgtgtctgcgggccagtgacagtgaggaccctgcaatgtgtctgcgggccagtgacaatgaggaccctgcaatgggcggcctgtaaggctctgccctggcctccgctgccttcgctttctccctccttggtgggtgcacgcccttgtgcttttcctaaaagagcaggtcctccgggcatggtggctcacgccggtaatcccagcactttgggaggccgagacgggtggatcccaaggccaggagttcaagaccagcctggccaacatggcaaaaccctgtccctactgaaaatacaaaaattagctgggtatggtgacaggcatctgtaatcctagctactcgggaggctgaggcaggagaatcacttgaatccgggagacggagtttgcagtgagccgagatcacaccattgcactccagcctgggcaacaagagcgaaactccatctcaaaaaagaagaagaagaaaatcctgacacttcagcctggtgcaggcccttccctccttctagtccctgccaagaagtgagccgggcccagatctcctgccgggcggggaatgagcacacacattcccctcttgggacagacagcagcagcagccctgttgcacacatgaggacgtacaggctcaggggccgtgggtggcagagaggctatcagcgccggactggcccgccccgagccagggtccagccccacagtcctgtccccaagccctggcccttctgcggtcactcccgtctgctgaatcccctactctgcccctggtgtgtggccccccagttccctcctgtgttcattccctgctaacctcccgtggcttcgcctccccagatgccctgagcacacagcctctccccttcctcccctcctagatgtgcacataggagccgcccaaaggctggggcagctagtggggcccctccaagggaagctgggccccgggcaatgccctgagccaccaggtcctggccctgcgtctcatcccttcttttttttttttgagacagagtctcgctctgtcgccctggctggagtgcagtggcccagtctgggctcactgcaagctctgcctcccaggttcacgccattctccttcctcagcctcccgagtagctgggactacaggcgcccaccaccacactccgctaattttttgtatttttagtagagacggggtttcaccgtgttaaccaggatggtctcaatctcctgacctcatgatccacccgcctcggcctcccagaatgctgggatgacaggcgtgagccaccgcgcccggcctcatttggttccttctgtgcaccacagtgtgggatctcgggtcctgggtggcacgtgtttaacctgaaaggaaactgcccctgcgctccccagcttgtagcccgtgggcctggctctgaggccccgactgccccacggcctgtgctgcgcatgctggagccaggctccggctctggttggcgcctcccgtggctttaatctgcagtgacctgggtgcttaacgagggcctttgcctctgcgtgtgcactgccttcttctgaggagtctcctcaagtctcccagctgtttaaacaatggggtcttttgtcttttgacctgttggtgccatcagccagcctcctgcacatactctccctacccttggctgccactcctgtcctgcccttcgccatgtcttttttcttttcttttttttttttttttttgacagcgtcttattctgccacccaggctgtagtggagtggcacgatcttggctcactgcaccctccgcctccctggttcaaccgattctcctgccttagcctcccaagtagctaggactacaggtgcccaccaccacacccggctaatttttgtattgttagtggagatggagtttcaccattttggccaggctggtctcaaactcctgaccttaggtgatccgcctgccttggcctcccaaagtgctgggattacaggcgtgagccactgcgccgggcccccgccatgtgtttttaagagcaggcatttcattcagatgaagcccggacactcttgggggttctgctcaggggctctacctgccactgaacgtcctctccctgtgtgggtggccatggcattcagcctgtgttgggccttgttctgcctggctactggccgctaggtctgccccggcatcctctgtgtcctgtgtgggcgcctctgcctcctgctccagcctgtgccaggcaatcctgctcaccttccaggagccaggcctctccccagggcctgcgtcctgtcagggtcagggacggcccctctgccatgctccggagtccctggtcccctcactccgttacgtcctgggtgtcacgggtacggccgggaactctgtcgtcttcactgtctgggccggggcctccctgggtgtctgctggatggagtgggtgcctttgggtccctgcaaagtgagcctgcctcccagcaccgccctgtcgttacatagccactatctttgcgcctgtttttccttcctttgactggttcctctggggttaattcccaggcctggtattaccatctctgaatgcctgggtggtttcagcgcccaggaggctgtgctgaggtatcttagaccatgtgggcaccgttcgctcctgcgcagccggctccggggtgccctgtttttgcggaatcctgcagggaaccccatgtaccctaagagtgctctccccagccactgtggcataagacaagcggtctctttgcccttgggccccatccttgtctggtcggcccttcttcatgggtccagcgcgggattgccggcttccttttcaggcttcctgggacccccactcagacctgcagctgggccagcgatgcccacccgtttctcctccacgtggtatacagaggtgcccaggctgctgctggggactctggagcccaggagtgagtctccttgaccctgagctgtcctggctatcacagctgggtcctgttttccccttcagcacccacgggtgtctttctccagtttatattgttatttttatttatttacttatcgagacagagtctcgctctgtcgcccaagctggtgtgcagtggcacaatctcgactcatcgcaaactccgcctcccgggttcaagcgattctcatgcctcagcctcctgagtagctgggattacaggcatgtatcaccaggctggacctctagtttatattattacagcctggtcagggagtcactgggcgtcgccatcctgtgggtgggaagggggcccaggcagaggccaggaggaggtgacagtcatgcgcttagaagctcaggccacgcccacccagccctgcctggtgaccggttcctgctgtgttgggagctgcatcccagaccttatcgccgggcacagaacttccaagccaggggagagggaggcctggaggggccccaatctctgaaggtcagcagtggcggggaggaggcttgcggtgctgaagggactcgggggacctgcagggaggtgttggtggtcagggatggtggcacctgaggggacttttggcagggccagaagtgcccagagcaagctccggtggggccctgcactggagggctggggtcgagtgaccctcttcctaagaccacccaggaggacactgggtgcagggtggccggagccctcacccccagtaggcagctgctgtccactccgccgacctgcctgtcacccagtaggcagctgctgtccactcccccgacctgcctgtcacccagtaggcagctgctgtccactcccccgacctgcctgtcacccaataggcagctgctgtccagtcccccgacctgcctgtcagccttttccctgtcaggccctgttcctaggagcctggagacctcaggggtggccttgagcccccagggtttttttgaggggaagcgccagctgctgtcttcacccttcccctagtgaggccaggctgtgcagggccacgtggaggcagtctgtgctgcgcccatcggtcgcctggcttcctgccgaccctcggcccccagccacctctggtctcgggcaaggcccctccccctgcccacctcctccctggcccccacgccaggtgggcagacctcctctgcggtttttattcagggggtccctcgttggctgcccactcttggagggctgtcctgactcaggcctcccctctactcagatcccctgagcgagggcctgggcgtgacgccgggagttactggggggccagagggggaggctcagcctgaatcaatgagacccaggaggaaggaggcacgtggacctgagggctggctggagccgctggtgacagatggaggagtaattgctgcttccagagcacagcgagctcgagctccctggagtgccagaagcttctgggtggacagacaggccgcctcacattccagaggctgacacagtcttccaggcacccctggggccagctggaagccatgtgcctccactcacgtgtcccgtgggtgtttggaggggaggctggccctgccatggcccaccccagcctgcggcctcaggagcttgcacatttgcaagggggtgacatgaagactgagccagggctgcggcgggcatccccctcagagaacagggaggagggggcacaggcctttttttttttttttttgacagagtctcactctttggccaggctggaatgcagtggcacgatctcagctcactgccaccttcgcctcccaggttcaagcgattctcctgcctcagcctccccagtagctgagattacaggcacccaccatcacgcccggctcatttttgtatttttagtagagacggggttttaccatgttgtccaggatggtcttgatctgttgacctcgtgatctgcttgcctcggcctcccaaagtgctgggattacaggcgtgagccactgtgccctgccaggcacgggcctttttatgagggaggaagccgtgccctctgccacctgcctgtgggctggggcctgcaggctcccagggccctcagctccagcgtggctgttggttattggcacctgggcctcatgtccacatggaaagtatattccgagctggtggctaagccacaagagcctgcttacctgtcccaacccccggctcacttgctgccatgatacgtctgggattctgggccctccttctgagcttggacacagacttgggacttcagcgctggacacaggtgctggatctggggtctcaatggcaccagggatggtcattctgacagccatgcccagagtctcaggagggcctggggccgcaggtggacaggcatcctgaccagagtctcaggagggcctggggccgcgggtggacaggcatagagtctcaggtgggcctggggccgcgggtggacgggcatagagtctcaggagggcctggggccgcgggtggatgggcatagagtctcaggagggcctggggccgcgggtggacgggcatagagtctcaggtgggcctggggccgcgggtggacgggcatagagtctcaggagggcctggggccgcgggtggacgggcatagagtctcaggagggcctggggccgcgggtggacgggcatagagtctcaggagggcctggggccgcgggtggacgggcatagagtctcaggagggcctggggccgcgggtggacgggcatagagtctcaggagggcctggggccgcgggtggacgggcatctggacgtcggctccactagtgtcacctctgcaaccttgtcccacccagcacctccctatggccctgctctgactgtgggctgagggggacccccatggctagcttgggacctccgctggacactgtcatagggcacttgtcgagtggccctgctgaggttcaggcttgggcgtgtgtggcccctgagcccagcagctgcgtgagccgtggggagccgagcccccagtcaagaaagctgtgtgctccttaccctgcctgctgggcttggggcctgcagggcaagggaggcggtgggatgggagccccgcctggcctggtttcttgttgtcgccgcactcctagctgtatgtagtcagatgaggctcccagtcctggctctgctgctgagcaccattgggccggtggttgggtgaatctctcagcctctctgagcctccagcacttcgtccttaagagtggaactggtctggacctcccaagggggggtcactgggtgcagagttcctgcaaggctgggcccggtgccagtagcggtcgctctgtggggcctttcgcacggttgatttttatctccctgtgagattagcgcctgccctgctctgcctcacggctctggctctccgggactgagaccacctgctaagtgacaggggcggaaggaccttgcaacacagagcaagacgtgatagggtggagctgctccgcccaggccccgcctgcagatgagagggcccagagcctcttaatcccctgcgctcagggcatgtgacggcagggagtctctccagtgccgcccaccggattcctatagttagagctcctcgcccaagcccccagggcacctcactggcctcccaggtgaggacatgaggcccagcgactctggtggtggctggtgtcacctatggcagggccgcgcggggcccagtggaggacgcctggcccggacctgactgctccgtgggaaccgtggggcgtgctcagggtgacttggtgcggctgggctgccagccggcctggctgagtcactggcagtgaaggcaccaggtggtggacgtgctcggcagctggcgggagagtacccccacccaggaccgtgttcaggctgagggggaagggagcggcccaggcaggcaccgcccatggcggggcccccgcactaacggccacctctcccgatggactgcagttctggagccgcccctgaggagaacgccctgctcagtgtcctggcagactctggactcttcaccgtgccatcctcgctacagcctggccaggcagccgctcccgtccccccaccccacagacaggaactgcagactcctaaaggtcgagccagctcccaagtccatagccagaaagtgatgcaggcggggcccggctggggggcaggtgtgaccgggtcatgggggagcgggctgtgcacaggcatcaggctgagaggtctcggggatacgtcctgggagccgcctgccaggcgtcctgtggggctgcctgcaggcctcctctggctttggcaggttctgtcccgacagaccgaggcttgggaagaccggattgtgcactctctgcctggagagagggtggggaggggctggggcgtgtgggcagggagaacaccctgggaggcatcagtcctcttccctggggacgggccatgcgtcaacagtccctcacttcctctctcaggggcagaggcgtgggggtcccgagcctccaggttggagtaggttaggagggcctcagagccccctgctgccccgccatgtggcattggaaggggtgtgaccccctaggggatctgttaaccacactccgccccatgccggcctctctgttccctctaacctgcgcaggagcagggccagctctgagctagcagaggtgacatgaggctcgtcctggccccgcccaccctggcccctccctgtttcccaccacgctccgcccaccagagctacatgttgtgtttgagcctcagccccagacctggctcaggtcctcaggaggaagcagtgacagccaggcccggggtcctctccctgctctgctccctgcgtgctgactggaggctgcaggtccccaggcttggccctgaccctcaggaatggagccggccgatgtgaggtgggggctccggtcatgtgcagtggtaggagaggaggcgggaccggtcccacagctccattgctgccgaggcgttccgcaggtctgtcttcatattggtaaggaaaatgcgaggatggtgtagcctgggcatcccagtccccaagtccgaaatctaaatccaagcggaaaattccaggcctgacttcatacgccaggtcacagtcagaagccagaattattgaaagtatgctctgggcgtggtggcctcacacctgtaatcccagcactttgggaggccatggtgggaggatcacttgaggccaggagtttgagagcagcctgggtaacatagtgagacccccgtcattatttacacaaaattatgtgactgcctttagggtatgtgtacgaggtatatattgaaatgtgagtagaattttgtgttgagtcccatccccaagatacctcattatgcgtgtgcaaatattccaaaatccagaaaatcctaaactggaaatacttctggcccctttggatgaggggcccccactctgcatagcaagtgtgggcgcgggtgctgctgtgtcctagcgtacttcagtgtgtggcctttgcagtgaatagggctggtgtcctcactgtacagatgaggaaactgaggcccagcttgctttgccaaggtgcatccggccccggccatggctattctggctccagatcccatggtctgcagccacaatactgctgtgccccggactgggccctgcagctcgcggtgtcgctggcctgcccattgtgggcaccgcccccaccccagactggccgaggcctagaagggagcagggcctggctgaggctgcaggggtggggacggtcagccagcccctcactgccaggaagggcgcatccatcctggcctctccccagggagaagggaggagcggctgagagggaagcgctcttgccctgtggacgagctcctgccccacggactagggagcccccgcccacaacctgcttgtcagggccacccgggacccccgggagttcggctgctcgctctgctgttaggaattggattagttttccataaaaacaggatgtggtgggtgagagggcagtgtgtccgtctttctcactcccctttttccaggaactgagcacgcgcataggttttagccagggccgtccagtcccctccccaccccccacagggaacaatccactctctgctcttaagtggccacttaatcagcttctcctcctggcccggggagcttcttggagccggcctgccgtggtgggaacagctatggggacaccctgccataaggtccagcagctaagctgggatgtggggggaggggctgcgaggcccaggcagtgtgccaggccgcacaagaggagcccagctcttgccccaccagctggcagccctggaccgaggttgggcccgtgaggttggctgggccctgggccctgggcccccctccccaggacacgactgtggtggcacatggctttgggggctcgtgggtcccactttgcagacctctgctttaaggggtctggtccacggggtcccctctggagggcctgggggaaatctcagggacctggggtctggacccgggggagggagcgggagaagcatgtgcgtgagtctcgtgctgtcagggagcccgggaagtctgcgagggcttgggggttgtgtcagggagtgtgggttttgcccttcagtggtggaagctggcttgagtcccctcgatccctcaaggctgtagtcctgactcggggctgtagggcagggcagggtggggtcaccctgaggcagaaggctcagcggagatgttctggcttggctctgcccctccctggctgggtggcctcaccctgtgacctttggggaccctggttcctctgagccaggggacagcagtgactccgccttcctaggtggctgaggatgacatgggctccccctgcaaatgtgggttctggctcagtggccaagtgtatgatggtatgtggctcttggggtcctgagagagatgggagaggagcaggggtttgtagggaagctggcggcttccaccccagccagtcacctgcagtgggggagttccaaagctgactgaagcttcgaccttgtggctggtccccttccttcctgcctcagtcattctggtctctgggggatcagggctggggggctctgggctgtgggggcctgtttttgtgacttaaagctctcccagcacagccccctgacctccttcctcatgggcaggacctggcccaggggtctcagcacagccacaggccagggatgcccttgcagatggccctggatggaattccagaactcagaaatgtctccttcccgtaaggatgtcccgagactcatgagaccgtttccttctgggaagggggaggaatggggagatgagtgaagagcgcactgcagctcaatccgggaagaagctaatcaatcaatcagggaagccaatcgatcggagaagctgatgggaaagctgtgcttggtagaataggctcgtgcgggcagagcagtcgcggcactcacaggctgactgtgaggaccttgggtgttactttgtgcttctctgcattttaaaaacttttgaaagtggagaagaggaaagtgaatgttctctgagatttcatggaaaggggaaactgaggcccactctagccagtttgggcccagggttccaacctggggtggccccggccctcgtggcctgaggtgatcgtccctgtggctctgagagcagctggggccgggtccccgttctggggctggtgatcctggggaagagccaggcagtgccctgcccacctagtggttatgagcccagaatgttgatttttttcccttggttgcttcatgactttgttgaatttccagagtatgtgtgggggcccccggcgtcccactcgccccagccctgtggcagcagagctggctgtcaagctcagtcagctgggcccagggccccggaggtaggtgggtgtgtgcctgagcttcccctttgggccctgccaggtgctgggagggaccacacaggcggcaggaactcggggtccccaggcctcggccacaccagcctggtgcttgttatatattgatatgtctctctacctgtgaaatgggtatttattttaaggagctgacgcacgcgattgtggggctgtcacgtctgaagttcgcagggcgcgctggctgtcaagagccgctgttgcagtctcgagtccaggctgtgcgctcaggccgggcttctctgtggcggtcctgggaattcctcctccgggcccttaggcgacactcccctgccttcagctgattggatgaggccctcatgccgtctgctgatttaaatcaatctcattttaaaaacacctgcacaccaaattgcgattggcgtttgactaaactgggcagcgtggcccagcagcgctgacacatgaatcaaccctcacaccccacccgcaggcgtgaccacggcacccacgctgccgactgagaacacagccgtgcgctgactcgcatgtgatgtctctggtcgcacctcctccatcctcagcaccctgttcagaatggagatagcggccgggtgcagtgctcacacctgtatccccagcaccttgggaggcctgggtgggcgggtcacctgaggtcaggagttcgagaccagcctggccaacatggtgaaaccccatctctactgaaaatagaaaaattagccgggtgtggtggcaggtgtctgtagtcccagctactcgggaggctgaggcaggagaatcgcttgaacccaggaggtagaggttgtagtgcgcgagattgtgccactgcactccagcctgggcgggcgacagagtgagactctgtctcaaaaaaaaaaaaaaaaaaaattagagataacgacttgggctctctagagaccagggagccaggctccaggcgctgctgtccagccccttgccagctatacgaggtgctcacctggccacgtgtccgggcagtgcttctggggtggcatccactgggagggcaagatggttctggtacaggtgcccaattctgtccccatttcacttactgggaactcaaggcacagagaggggagggctgagctaggaccagaccccagtctcctgcagaagtacacagacatgatgtacgaccatcctggacacctgccctgagattcccctccctctcctgccctgtcccagtggcctggggggaaggggaagcaaggttctgaagggagtgtgaccagacacctgcccgtgacaccccctctccaggctgcctccgagtggctggtgactcccctcctgcctgcgagggaggtggccaggttgcattcctctctgagtgccggggaagtccctagagagcaggccagcctgtgactgggccctggggcagtctagacaggccagactggacaggccagggggctgggtgccgctgggtaaatcacagggtgagggctctgagtcagcaccccatcttctgtcctgggtccagcaccgctgaggacacagtgggcagccgggtctgccagggccaggtagctgtgttgagaaggcagtgctcctgagaggcggctaccgggaggttttcaatggccaggcttcttaggaagcccttgttgcctctctggggtgagttgctggggccatggttggagtggtcgccagtgtctgcccctggtgccgagggcggagtcctcgttttgggaggtcacggcatgatgctgggagtcaaaggcaggccgtggcaggggcattccttttttctttcttttttttttttttttgagacggagtttcactcttgtcgcccaggctggagtgcagtggtgcgatcccagctcactgggatcccaggctcccaggttcaagcgattctcctgcctcagcctcccgagtagctgggattacaggtacgtgccaccatgcccggctaatttttgtatttttagtagcgacggggtttcgtcatgttggccaggctggtctcgaactcctgacctcatgatccacccgccttagcttcccaaagtgttggcattacaggtgtgagccaccgcgcccggcctggcaggggaatgtcgacgcgtgatctctgcctggagagcacgttcatgtttcccagaggacactttagaacatggcgcctgggtttggatgaacctcagcctaagaatctacctgctcaggatccagcgacgctggtggtgtggacttcagctctggagaatgggttatatggaacctgggcgccgggagggcattgccacgtgcttgctgctggggcttcaagaggaccccatctcctgtggccgagaccccgtgtctcaaggcacatcccctttcgtaccccgccccaccctccgcagcttcatgacctctgggtttcccccaggaccttcgcatctgatgttcccagatccttcctgccactgggtcctgctctggtgccccccggggaagccttccctgaggacccagctcagcatctggtgctaactgtggctcatcgtgcactttggcccccaggaggctgtgggctcctgaaacccctctgaaccacgggcttccaaccacacctgaccccctgcgtgccgaccctgtgtgcaggtgtacaggtgtgcctggggcaggatggctgtgacggcctcacagagcccggagagctgcctcctagcttccaaagccttcatttcaggaatcttaccctctcaattagtctggaatgctgggggcggggccagctccaggtcacagagcgaccttgtttacccagaccttaacatcggcccttccatgctaatcaatgtaatcattgtggtccatgcggcctctggaatgtgctgtccactccctgggtcagccagagagtgtcagggagcacctaccggctgttacccagtgctgccctgacctggttcttcactctgcacatttgtatcacgccagaccctggctggcagctccaggtgacgaggcatgtcagtgccttcctgttctgttctgttttgttttgttttttaaatcgggatgaggccttcctgtgttgcccaggttggtcttgaactctcagggtcaagcgaaccttctgccttggcctcataaactgctggattacaggcaggagtcaccatacctggcccactgctactttctagatgaagagacagaatcccagagaagaagcaggggtttggctgctggtctggagccggttctgctcacctccagcttctgccttgggccgccctgttcacacaggagctgctcacaggctgagacctcgagcagggccctcctagaggaactgggcccccgtaagtgccctgagccgccaggagccggccctgcgtctcatccctatctccggaggacattggctgctagctcaccagctggcccctgggcaggcttgaatcatgacctggaacgccaggtgtctctggctctacccctggacctgcaccctgtccaagtgccccagggccagactgttttggttgcaccctctggacgggctacccccatgatggctgctcatggaaagctgtggttcttagggagctgccaattcctagtcctgcagcctggagctcctgggtataaggtgggggctcgggcgtattggggactgggggtctcaggaaagagcctgtgggacctgtgaactcatagctgctggccgagggaccaccttgtggctgtccttctcagctaggcctggtcagggcttgtgtgcagggcggctgaagctgtgggaggccacactgtccacacagtgccctgtaagcccaccgtgcctcagtttccccgtctgacaagtggcacaacagaagccacttcctggcacacagctcagggtcagggccgacacagcacttgtgggctgccgggagacccgagaggctgccccttccttgccttggctgccacgggtgacctggcaaacccctctggccgtggcaccactgggggtctacccttggcagtcagggttggccgcttggctgggagccccttctcctccccagacacatcctctccttggggctggagggggtcctgccgtccccgggattgtcgagcagcaggaatccaggagggcagtgcctgcagctcagatgggggccagtgggcagggccgatccaagggtggcaggaaagtgcccatcactgacctcaggtgggggaggccatggtgtgtgaaggaaggagagctgagtgggaggtcttactttgtcaccgcccctctgagtgcctgctgtgtgcaaaggccagcgggggccctttcttcagctgggctctgcccagacccctgagctctgggtgggccgggagggagaccttgctgctcacagaggtggctctgcctgacccagctcccttcccagggcacagtgggggcatagggtcggctccgtcagacattccgggacctgcgtccttcctggggccacaccctctacccactgtcccccacctatttacctgtatctgcgccagagatggctgcccagataagccctggtttcctccctttctggagaggctgcgggggctggcgaggaacccacctgcgcagagagtcaggggattgctctgtgtggaacgcaggcctcacccatgccctggaatctgtccccttctctgttgctgagggatgagtcccagatccctgaccctgcaggggagcccagcacagaaaaactctgaggcctccacaccctggcagcgctgctggtcgtctgtggggaaggacaggccctgggagggagggggaggtgcggagggcagtggggagggtcaggaggaagtgggggaagggccacccaggccccgatgtgggggatgtctcacgcgtggggtggggcattctcatctctgcttggtctcctgccatgctgggggtcgttcactgcggaccccaagtaccatgaagatggggatgggatgctgagcagcatcggggagaacgcaaaggcacctcccagctcacccgcccccaccccgcaagcacaaccattgccatggtgtggggaccggaaggcggcggctttgggaccagactgcttctgcctcgggccgtgccgctgggcctttggtcagcaccatcgtgccctgcaagtcactcttagcctggtgccctcctgggcagggcagtgccacaagagctcaggcccagatgtgcaggtgccactgttccacccaccagcaggtcacctggggaaccctcccctcctgcagcctctgtgtgctcatctgtgaaatgggcgtggtggagtcacccctggcttgtgggagaatcccaggggctgatgcctgcaggaccctgtgggctttgccccgctccctgggcagagacagttcccccagtcccacccacgtggctgtgtgcagcaggtgcctgctgaccctctgttcctgcacagtcaccttcttcacagacggacccccaccctgccctgcaaacccctccaggggctgcgggctgagtgtgtccgggagggtgtcctgactctccacgccagcaggtctgagagcagatggctgtggcaggtgcggtgggtgcccagcccacagcagccaccaggcctgcaggaccctgccccgtgtaggtcagatgagccataaaactgagtttcctggacactgagctaattaaacctggacaccgagctaattaaatggtccagaagctcctcagtgccccaggctgctggccgggctccaagtaggtgaggaacattccgtttacctcctgctgcgtcgaaggcgggtggctcccctcgggcccctgcctgtcccgggccccctgggtgctgctgcgtcaaaggcgggtgtctccgcttgggaccctgcctgtcctgggcttcctgggtgctgctgcctcgaagacgggtggctcccctcgggcccctgcctgtcctgggctccctgggtcctgctgccttaaaggcgggtcgttcccctcggatccctgcctgtcttgagctccctgggtgctgcctgccctgtggccacgtcccactgttgcctttggacaaagctctcctagggcctggggttctcccatgagatgacaggggttggttcaaaggtctctgaggtttttagagctttgtgaagtgttctagaatccattttctctctcattgctggacagagagtatagactgggctttttcttgagtttctctcaactcttctttgtccatgaggcaggaagaggggcttcctcagtcctcagcttggggtggattgggatggacagagaaccgtgcaggatcccagcctgagacggtccagcgccccgggggagcggagcccgtggctcagccgtctgtagccacggccggggtcactgtcactgagggcacggaggccccgccggcgaggtccgaggtgggcgtgtggggtggtgggcgctggaggcaggacctctgcttgtggagggtgggccaggcgtggaccaggtgtcacgtccgctggggcctttgaggggcaggtggggattggcgcggagaaggagtgaagcagctggggttttgggagcctgtgcggagcaggcccggatgccagggtggctggtgaaggtgggcctggccgggctcggcctccattgggggagccccccaggtcccctccccacacagcttccccctctgtctgctgtccaaggctcctgtgctggcactctggggtggtaggccatgcagcccgtgtgaacctccatagaccttgctgtgaacgctgcacggggcgtctgggggcgggctggcttccccccgtcccctgggccaaccttgccagccttcttcttccataaaagtgggatccgtctgagccctcatggcccctctgggtctgggtttgctttttgtgctctgcctggggtcatggcagggaaggcccagcaggctcccttgcagagcagggcagagcagcggccgccaggagctgcctgtaccacgtctgctcttcttcctctcttcctctgcccggccccgcccaccgtcgaaagcactagcacgggagtgtttatcggtttctcttccaagccaaataaggcagagagcgctcttgcaggagttggagcaggccagggggagggcagctggggcttggaccaggaccccggctccctgtagctgccctggcctggcagctgtcatccgcgaactctgatcctcgcctgccctcacccggcccctttcagagcttcccatgctcagctggtagcaaaagaagtcaagacctagcaaggtgcctgccccggccaggaggcccaggttctagccctgcagctgccgctgtgagctctggggactcagacgactgatgtccttcccagcttctgtttcctttgatgagaggaaggttggaccaagcgacctgccctgcttctgtctgtttcctccagtccttttcctttttgaccagttcacttgttgataactccagcaacgcatgaaaacattccccttgtaaaaaagggaaatgccgtgggtaggggggaccaaggacctcttctgcaggagcagacgcaacagcttggtgtggatcttcctcagcctttttgtccatcctagacatgtggaatttcagttttacacaaaaggaatggtaggcgtctgactctctgtggcttgtatcacttggcagtgccacagggattggtgcatgtttgctctctcatgctatgtgtcaggcactgttgtctctgcgacaatcctgtgaggagggagttatggttcttgatttatagacgaacaaactaagacagaggagattgggctcatctgtggtcatctcagcagttgtgagaggtcagggccagagcccagtggtgtcattctgcagggcacatacagacacgacccacagggttctgccgcaccccctgccgcttggcctgctctgcaggtgggtgccgaggctgttcctgcatctgctgtgtcttgtgagtccgtgggtgcctgtggagtgggggcagtgggtttgcatttccaagcactgcctctgtcataggctcagggagaagcagttacgcatgagctgctatgagccacgggtatgactggcatcacagcaggaggagccctggggatggctgagtgacgatgggtggggacagagctcacagaagccagggttgcccccaggccaggccactgctggggccaaggctgggatgcagcacgatgttggtctgtcagggagtggcaggaagcccacagggtcagctgacacttaacccagagtggtgggagccttgaatgccagactgagggccacacagttggtctcggtgcacaaggacttgggctgagagggctgttgctcctcaatggccatctccttgtgggaggagagtccgggcagccgcttccaggaacccacccatgcttgaaatagtgggaccacagggctgaggaggctcctggggattcctcccaggattcagctgctctgtccccaaccccggagggcctattcgtctagccctatatcctctctgccagttaggtcagggcagagaagtagaggcacagagggtggcggaggggtgtccagccaggcctttctgtggcaccttctgaggctggctggagtctgtgaaatctgtgataactggcccagacccttcctgcctcctggggcatctgctggagctgaggctgctgggggagtctgcctgtgacttgagtctcttgctgggccatgctgggctcagttcgcctgtctgtggggtgggatggtgccacccttaggttgttgggaggacccaaggagagtgatgcctccagccatggcagctggcccagctccggcccgcagcctggctccttcagggccaggaacccccaggtcatggactccaaccctcgggctctcctgcttcccaggtgacgaagcttcaacccatgggctctccttcctgggtgtggaagccctggagaaggtggagatgggatctcagtccagcccattggctccacttcctttgagaagctctttcccccgtcttggcccccagcccttgttcaagcacctgtccctctcctgtctcctaggccccgtcagtccttttggagacctgttcccacccgcccctgctctcacaggctgccagtgtccacacactctcaggcactgtcattggagccttttaaccacacgggaaagcaggcaggttaggtaactaccccccacccccccgccagcaacgccccctgcatacccagggcctgcaccaggtgctgctgcgctgccagctctctcgaggtccctcctcctaacctcaatgcatcgcgtcttccagcccccggctccgagggctcagcctccaggtggtctaacctggagtctaaacctggattgccatctgcctgcctcagcccttcccttccccagggaccgagggccagtgcggctccccagccacccattttttgtttgttttgagacggagtctcgctctgtcgcccaggctggagtgcagtgtcctgatctcggctcactgcaagctccatctcctgggttcacgccattctcctgcctcagccccccaagtagctgggactacaggtgcccgccaccacacccggctacttttttgtatttttagtagagacagggtttcaccatgttggccaggatggtctcgatctcctgaccttctgatctgcccgcctcggcctcccaaagtgctgggattacaggcatgagccactgcacctggcttgtttgtttttatttttattgttttttgagacggaatcttgctctattgcccaggctggagtgcagtgacataatctcagctcactgcaacctccacctcccaggttcaagcgattctcttgcctcagcctcccaagtagctgggactacaggcacccaccaccacacccagatacattttgtatttttagtagagatggggtttcaccatgttggccaggctggtttcgaactcctgaccatgagatggcgggcttgagccatcttcccgcctcggcctcccaaagtgctgggattacaggcgtgagccacggtgcccggccccagccaccagttttgtacagcactcaggtgggtgtggttcctgaggacatgtcacacgagatccttccggtccattagcccaccccgccccatcagtggccagtgcggccattgtggggtccagagtcccgaggcagaacgactgactccaggttgtggaggggcctcttaggatccttctaggcacccaggttgggcaggtgcctccatgcctaagactgagctcctggtggacatcgggctgggtgtgggcgtcctgcacaccctccctgtggcctggtgcccgtgaggagtggacagtcaccccgagagcatagaccaccctcgaggctcccagatggcggccgcagagccccacttccctgcctggtgtgtgataccttccatggctgaggacacctctgactgctcactcctgtgaccctggacccaagtgctgggccacacctggactccaggccccagtccttccttcttctgctgtcctggctcctgggtggcccccgccctccccgctccgaggctctgccctgtgctgggaacccaggaattgccaccccctggccccggtttctcatgtcaggatctgctggccaggttgactcctgagcgccactgtctcctcagacttgctggtcccacccggtccccacctgtccaccgcccgaggctctcctgtgaggccgctctgcagctcttggcctgggggcccccggcctctcatggtgccttctgcactgtcccctttggagatgggagctttgagggtaggcgccaccgttcggtgaaaaggcggtataagcaaagcaacccccagatactgaaggagccgggaaataaaaggaggcagacagagcaagtgtgttggtacccgcctatttactggcaggaacttacagacggaaacatggcctcaggcagcctccaggcaggtagagctctgaacccaaacgcccgatctggggctcgtatcgtgccctgggaggaatgtgcaggtggctgggaacgtggcgggtggggtggccagattcctgctgcagcaccgggtttgtttaggaagaaacgtacgaggactagacactcttttttttctttcctgcacgtgaatgccagggagtaggtgctcctgcaggaagatgatgcatcaaccagccagtctggaggcattccgagacccggggttaatcagcagattagcatttaaataaagttactctgtccccacagccagctaggcccgagccacatggcgggggcggcgggggcggctgttcgccgcatcccaggcttgctccatctcctcgtctgcaatgtggaggtgacagtcgtggtcctcggctagtgccacaggactgtacgggacagatcacccagccccgtgcctgcactgggggcggcggtgacgtgatggccgcggcggtgacctgatggccgcaggtgtgtccaccctctggttaggcctctcctctccctggggcgcaacaggtgtcctcgggtgggaggttcagctgctgggcctggggccccagctttcagctcctgtgcaggcgtggagcgggacggcacgccggggcgttcgttcacacgatgcccccagctcgtctctgcctgctctgtgatgcagcctcagctcctgtgggtcccgggtcacccacccgtgtgggccgcagtctttctgcaccacgtcctgctctcggccagcgctggcctgcgggccccggcagtgtggggtcacacacaggccgcaccggctgtttcacggagcccccctgctcaaaatcctggccttgggtctctgtgtggctcctggagggagcatgtatgaggtgtccaggttgtgaaagccccatccggcagccctccctgtgacagtccttcaggcctcgtcaggcttggttcagcccgtgttagaggaggccagacagctcctcctcgatcaccacagggaacgagggttgggcagtttctcctctcccacccgggcgtgggtcagctgcaagggtctcatcccaggaactggatcctggcaggggggctcaggcatctgacatcccctggcaggtcttcctatgtgccagtgggagtgggtggtgtccagccctctttgtggaatgttccggttgagtggtgtgtggtcctggttaccaccatgtggcatttgtggctttcctggccagcacccactgaaagcagaggccagcccagcccttggacacccaggggcccagcagctaccgtgggggcagcctctcccctcctgactgtgctcctctcagctgtaggatgaaggtctgtggatgggtgatggctgggctctctgcttcccaaggagctggatgtgggccatagtcaggcgtgtgctggtgaggcgagggggttgggggttgggaaccacagctctctctgaccccatcccattgcagctgactcaggaaaaggcattacccccacggggtcacaggcccagcctgggcagctcctgatgggaacatgggagccacagggcagggcaggctcctgtccccgctccttggattgtctcaggccccgcctcccctttggctgaatttcttccttctctgcctgagcctgatctgcgaagcttccgacctgccctgagttggagccagtcttcagggccaaaagcaatgccgtctcctccaggcagcctgctctgaagtctccccctctggctttagcccacaaagtcacagctgaggccacatcaacacacttagaccaagagcactgccggctgcatagacagcacaggtagatccaaggagtgtttcgaaaagccgtggccctgcggccgggatgcccggacctgggtcctgaccactcttgggagctcctcccctctcagagcccatcccatctgaaaatgggggcattggagcattggaggagtgctcctcactcctgccctgctagccggctactccgaggaccccaagaccagatgatgggagaagtggtgggctgggctgtgaccctgagcccccttctcatcccacccgtgaggctgcccctgcttggtgtctgtggcaaggctgacagaagatggcgccagggaccagccagggccctcctgtctcagcccgtccttgggtgagtgggtgtgctctgggggtggcaggtgtgctccgggggcgggaggtgtgctgcggggggccgggtacgctctgggggggcgggtgagctctggggggctgggtgcgctctgggggggctggtgtgctctggggatggacaggtgtgctctggggggcggggtgtgctctggggatggataggtgtgctctgggggggcggttgtgctctgggggggcaggggtgctccgggggtgagtgtgctctggggggcggtgggtgtgctctgggggcggtgggtgggctttgggctaagtagccctcttcctctctgagccttttccttgtctgtgttgttggtgagtggccgccacctgggacaagccttagctgtctgtccaactgtccaccatgtccccggaccctagaccaagcccgctgcctggtggggcctgcttggtattgaatgaatgaatgaatggatgaatggatgaatggatgaatgaatgagtgaacaaactctggggggcgtgttgctgatcaccccagggcaggcccgtggtgggtcatggtctcaggcaggggacttgccctgccgggtgacgttggcctgccggggaacccctgccaaggtgcctcccatgcccccatcgtcctctgcctttgagtccactctgcccctcaaggttcccctctggctcggccaggggcagggcggtcagggaggcctccagctccagcccctccctcatctccatcctcagcccctgcacccgcctcatctccagccccagccccttgcagcctcaggctcgggcttctgagaatcctttgcagatttcagggctggttatttttttattctggcaaaatatacatccataaagctttgtttcctgagatgatctcaccctgtcgcccaggctggagtgcggcagcgtgatcatggctcactgcggcctcagcctcctgggctcaagtgatcctcctgctccagcctcctgagtagctgggaccacaggcatgcaccatcatccccggctaatttttgaatttttttgtagagatggggtctctgtgttgcctggtctggtctcgaactcctgggctcaagtgatccgcctgccttggcctcccaaagtgctgggattacaggcctcagccaccacacccggcctaaagtttgtcacgttagccgcattgaagggcacaggtcagcagcggtaagcatgttcgcattgttgtgcagccatcactgacacccatctctagaacttcttcgttttcccaaaggaaactgcacgcacggcaccacctccctggcctccccacagcctcagggtctggtgaccttcatccccaggacctacgcttctgactccaggagccctgcctggtgtccacaccacactaaggtgccctgccacgggcttcgaacccaaccctggtgcacttgttgagcattggacgttcctggcaggtcagaccgtgagctcctctgctctgacggatgcatggacggctgccacctggctggacaggtgcagaggggaggtgccaccttctctggggacaggactggggcagaactcacccggctgaaaagtgcagctgtggagaacttcagaacttcagaagcctttgctgtgaatgcattttcctcctgcctcttgggccagatcttagggattttttttttttttaatgaaaatgtataatcgtcaaaaaactttagatttaaaagcatggagccgatggctatgcttggtgcgttaatgggaggaagaggggatcttaacttcagggagggctcctgggggagctgggggaggctgctgtgtccagcagggcgggcggtgccccccggagcccggcactccgcgatgtgtgcgctaagccgaggccgccttgagcccgtagcagcgccgagcgcgattctttcgtgtctgcttccgggagggtggaagggtgaagctgttgagagtgggaagggaggggctgtgcttcttgagtttgccgtgtgcccctgaccctttcaggtgcaagcgtcagctccacggtgccaatggggagatgggtctggaggccccaccaggctgtgaaaggcccggcctcctgtgctgtccgtggaggtcggggtctccctctgccctgcgcctcctctaacttgccctgcgttttccgtgcccctggtctagggtgaggctcgggtcttggggactgaggggcaggtgggtgtcaaggtgagagctgggctgggctgggcctccctccagaagacttcccggggctcctttggcaccaaggatggggcagatggtggcacgcatggcctcccctgggcacagagccagactatggagggatctcgttggctacatcctggctggggctttggggtcatggtgggagcagcagccaggggtctcgcttggacacgggtctgagctcactgtggcctccttcctcagcctcctgaccggggtcctgaggctggagggtgggtggagggtggagggtgggtggatagtgggctggaggatgggctgctggcgccccttaggcagcatgaggcgcctttccagtcccagtgtcctccctggtagccccggaaaggctgagtggtccctcaggtcccctgcgaggagacgacatcaggccccacccctcatctcctgacaaacctcacattcctaggaggaagcaggcctggctagcacgtggcaggcacagcctccctgactccagaccttatcagatcagtgcaagtgccagggagaccccgggtgggagctgtgtggttgggactgatgcaggcactggccggactctcaggatgagggtgggaggccaggccccgggaccttctgggacaggcaggagggcagacacgggacatggtgtgggggctgtggcgagtgcggccacgcagagcatttgggctgaaggcgcgctgtgtcccactgccccatacgtgtgcgggggctcccagacacgtgctgggggcaggaatcatagcatttttttgtttgattgttttttgagatggagtctcgctctgtcgcccaggctggagtgcagtggctcaatctcggctcactgcaacctccacctcccgggttcaagcgattctcctgcctcagcctcctgagtagctggaattacaggtgcccgccaccacacccagctaatttttgtatttttttgtcaagacagggattcaccatattggccaggctggtcttgaactcctgacctcaggtgatcctcctgctgcagcctcccaaactgctgggattacaggcgtgagccaccacacccagccaagaatagcattttctttctttctctttttgaggcggagtcttgctctgttgcccaggctggagtgcagtggtgccatctcagtgtaacctccgcctcccggtttcaagcaattcttctgcctcagcctcccgagtagctgggattacaggtgcccgccaccatgcccggctaatttttgtattttcggtggagacagggtttcaccacgttggccaggctggtcttgaactcctgacctcaagtgatccgcccgccttggcctcccaaagtgctgggatgacaggtgtgaaccactgcacccagcccaagaatagcatttttaaaggaaaaaaagcaaggtagaaagatgggtcaaaaccccagcgccccgctgggaggctggtgcccgtgtcgtggcgtgtgtgagatatgtacggaagcgcgaggcggcttccacggctctggctcagggcagcactcacagggcacacggcctcggcggtgtgacgagctgggccgtgggtgcgctgtcctctggggtggggctggccatcctctgttggcgacctagttaacgcccatctccccgcagcctgcctgctccgcttcagcggactctcgctggtctacctgctcttcctgctgctgctgccctggttccccggccccacccgatgcggcctccaaggtaaggccaggggaccctgcccacgctctcagggtgggagggggtgggcattcgtttggggccaaaattcgtacagctttcctcacctttaacagcccggggtggaagatgaggggtcccgaaataaaatttttttttttttttgagacggagtctcgctcagtcgcccaggctggagtgcagtggcgcgatctctgctcactgcaggcttcgcctcccaggttcacgccattctcctgcctcagcctcccgggtagctgggactacaggcgcccgccaccacgcctggcttatttttttttgtatttttagtagagacggggtttcaccgtgatagccaggatggtctcgatctcctgaccttgtgatccgcccgcctcggcctcccaaagtgctgggattacagacgtgagccaccgcgcccggcttttttttttttttttttttttttgagaccgagtctcgctctatctcccaggctggagtgcagtggcacaatcttggctcactgcaacctccgcatcccaggttcaagtaatcctcgcatcccagcctcccaagtagctgcaattacaggtgtgagccaccacgcccagccatcaaagtttcttattttcattgtagaccccaagctaagctcagaatcttttctgatccgtgttgcaggggctgtgaggggcgaggagggtcatgggggtggagtctctgtgtcccttgggaggccacagggagtggatgggccccaactgcccactctctgcaggctccaggcacgtttcctgcctgctgaggctctggcagcctgggagttccagccgcctggaccgccctcccgctgcccgctgtggggttggacctcagttgactttgatccacttgtgcgggggggggggggagctgagaccccccttgggaaccaccttggggtctgagctgggccaggactgtgttgctctcccgtatcctccaaacagagggcggtgaggccacgtgccaagcctgcagcctgggtgacgagggtagcagcaaaccctccatcctgtgacatgtcacctacctgccctgtgggggccatgtccattttgtgcccacgttttaggagagcgcgttttggagcaaacactcagagacctgtccccaaaggccatgggccaaacgtgggtggggccgggccctccgctggtctttgggtttggagggtgaagagccactgaaaggaaggaaggtccccagggagggcaagtgtaggcagcaagagaccttccccagcagagggcagagaggaggccaagcaggaccagggggcgtgactgtggagacaggctcttgggtgccgcccttctgggaggtgctggacgcatcaggctggcagcgtcggggcctgtccatggctacgaggtgtccaccgggctgcgtttttccaggggactctggggaggatctgcttcctcttccagctgctccaggtgcctaccttcctgggcttggggcccctccctccatctccagagccggtggggtggggtcaggatcacaccctgtctctcctgctccctctcccctgcggtaaccctgtggcttcgtcccatctgcctccccggctaatccaggccgacttccctggtttaaggtcagctgttgggcagcccccatttcctctgctgcctggactcgcctctgcatgtgagcttctgcgttcacagattctggggactggggtgcaggcatcattggggtccatcgttctgccagctgcaggtggaaggtcgctctccggcctcgcgacatctggctggaagcatcccagaggctcctcctagctcgtggtgctgtggggtgggcaatggtcttttttatttattttttttgagatggagtctcactctgtcacccaggctggagtgcaatggcacgatctgggctcactgcaacctccgcctcgcgggttcaagcgattctcccgcctcggcctcccgagtagctgggattacaggcgcccaccatcatgcctggctaatttttgtgtttttgtagagacaggggttttaccatgttggccaggctggtcttgaactcctgacctcaggtgatctgcccacctcggccgagactacaggcatgagctaccgtgcttggctttttttttttttttcgagtgggggtcttgcttggttgcccaggctggagagcagtggcatgatcatggctcactgcagcctccacctcctgggctcaagcgatcctcctgccttagcctaccaaagtgctgagattacaggtgtgagtcactgtgcccagcgcaggcactgatcttggatcatgggtagaattcaggggtgcagagaggcattttgggaggggcccagtgcggccgagtgctgggagtcgctggggcccgaggcccaaacccctccaggatgcatggctggggtgggtgggcacccccactcccggtccctatcctgggcctcccctttgctctgtggagccggttctgtctgttcccaggcccctggcgtttcgccgtttgttccgtaaatatttcatgcccagggcgcaattggaaacactttcctttaatcagcagtgggggaaggcaggcgcccagccaggccaggggaggagctggggtgggaagatttggagaggacccgggaggacttccctgcctgagcctcgtcagaggcccttcagagacggagatgctgcccagttttccgggagggagagaggaaagtgtgaggcttgcccgagccgagtgcccggggctttatgatttgtgcagctgctgggcttggcgtggccctggtggaagctctgacgccatctgagccttggtccccttgtaggcgtcagcctgatccttggggggctggaggttgagctcagactgaacagggaggagaggggtaggggctggggctggggcagagggaacagaccctggtgctccaggcagggctgcaggcagagccacagggggtggctcccagagacctgcttgtcatttagggactcagaagccccatcctgccttaggataaaacccacctccaagactgaccctgctgtctggagagagatcaccccccacctcttaccatgccccgtgcagggaggccctgccaccctctccagcttcactggtcccccggctctgccagccttgtgtagtctccatcctctgtctcaaaaggcccctctcgtggccctaccccgcagctcagccctagctgtcgctgggtgggcctcatggctcagcctcaaggcaccctgggtcccttggctggtcccatgcacaccccatggatcaggttccagggggctcccatgcacaccccacggatcagactcagggcaactccacactccccacagaacaatcaggcccagcccttgtccggcaggtgtggggatggtggctatggaccacgcatcaggatgctgccgtggtggggcccctcagcctttgcaggccgtgccggggtccaagggctccggctctgggggaggtccatgctctgtcaaccatccggtctgcaataccctggggcctccccgccccttcctgtttcctcctgagagccctgcaagctggagggggagccccggcccctttttttttttttttttttgagacggagtcttgttctgttgcccaggctggagtgcagtggcgcgatctcaactcactgcaacctccgcctcctgggtttaagtgaaagtgattctcctgcctcagcctcctgagtagctgagattacaggtgcccatcaccacgcctggctaatttttgtattttcagtagaaacagggtttcaccatgttggccaggctggtctcaaactcctgacctgaggtgatccacccacctcggcctcccaaaggtgtgaaccaccacgccaggccccctgcaccttcttctgggtgggcaagagccatgctgagcccctcccctgccatggataggggtggcaagaatagtccattccccatgggctccctccggcaggcagcggatgggttggggcccacacagtggttcaggaaaggctgaagggcggttgccggccagagctgggcgggtccctggcctgagcctcctctcactccctttcccaggatggaaaggccactgtgagctgggtgtgtcctgccaggagggagttggagtcgggggcaggagacccactgggtctcctgggctcctggaagaggcagcagtgggtccctgcggaggtggcctgatccccggctcagcctggttttcccagctttgcatttgggggtgggacctgcagggggagagtgacggggtgggcggggacaggggctgcaggtggaggcacgagaaagccacccgaccccttgaggtcatgctgtttgcgtcggagaagagggccggtgtcagggtgccccctcggcctctgtgtgcacctccccgctccccgccccgtgcaggtcagcagagcctcgatccctctgcacgttccggccccctcccatctccagcagcgtctcttcccggggtgcccctcggactctcagctccctagtctgatccagcccaggcagggagcgggcgtgccacgtggccaggagtggcctccaggcccagctgcggccccttcgaccctgcggagggatcatggcccagctgttctaacatggccagaccaggacactctggccggcccgagaactgaaccgggaaggaggcagggaaaggggagggaggaagacaatggggaagcagtgacatccgagatgtagccagagacggacatcctggactgtcgtgcagggcaaggcgggtgggcgggagccaggtggcctgagagcccctccctgcccaggggtctctggtcaggcagttccttcccgggctgctggatcgtgtgtgcagggaacccgtcagcctggctgcccaagggcccagatgtcctctcagtgcccgggggtccttgacagccccagcaggagccccacgtgcctgtggggcaggccccgcaggtccctctctgtaggactcagaataccttctccaatgccacgtgctctccctgagtgcccagtgccacagagggccccgctggcgaggttacttcaacgtcaggctagagggtgcagcgagcaggactcacagcccaggttcccaggcaggtgtggcaggagcccattccctgggtcggtgttctcatgtcaccccacggtgaccctgctgtacagaggcgggggcgcggggcccctggctggtgccctcctgctgccagacttgtgctctgctgaagaggggctggcgcggcaggtatgagcccggcacggaggtcgtgagcagtgagaagcctggcacctatgggttcggggcagggaggccctggaaggtcctgtcctccgggagccctgcacagcaccccctggtggtggctcctgttcgtgtcggggctgcagcctcccttcctggacggttttccattcctccttcccactctccctgctgctcacccctcccatcctccacaggtcagaggtcatagctgcagggtcagcaggtcagggcctggggcaaccctaggaaggtgtgagtgtgagaagctggtccaaacctgcctcggctgcctggtggtgcggacagggagtcctgggcatccgtgagggctgcttcacggtcaggcttagggatgtgcagggtgacttggacgtgggtcatgagtctttgctccagaaagagggggctgagtgtccaaggccaatcccgagtctgtcacctaacaccatttgtgctcaaaaaactgaacagagtggacacaggccctgagtttgcgccccgaggccgagagagggcagatgtggtggtgacattcaccaccttggacccagacccagatgctacctgtccctgagctccaggaagttgtgagaagggcctgagctgttctgcactttctcgtggccgggcgtggctggtggtgcaggagttgctgccccagggtgagggcccggaagctcctgccagcacgtgccggggtggaaagggaagctgtccacagccctgtcaggactcagaacccggtgggtcaaggactttggtccggacccctgctgaagggtgagctgtccacatgtgcgccgagagcagaggtgaagccagggctcctgagtgcccccagccacagggtgcgcccgcccagcccctgccctgcagccgaaggcctccctgctggggggctgagtccagtggggccacaggcagctgggagcaggacaaggctgccaggcaaccagatggtgctgccgcttcctgccaggtgtgggtgcacagagagagagaggatgccggtctggggcctgacctggtgcacagcaggtgcctgaaatgccagggtggccatggggactgggtaccatgcataggccatgcatcgggatgcaacttctccttgcagcccctcagccccagggaggcagctgcctgccccacttttctccagagccatcatggccctgctcccaccccagccacggctgctcaggggcgctccgcatgctctggtctccatccacctgcagcccccactggggtgaggtggagcttcttgcctctccttgtgtctatttcctctgcttccacaactgaacggtgacaggtatttgctggatgagggagcacaccccaggtggtttcctctgagcctgggaggccttttcctgcctgtgggccccaggcccatcctgctgccacccccaggaggatgcccggctccttgtgacaagagtgaccctcgggaggcgtggggagtggggctggccggcctgcctgatggggtcctgagtccatggcgggtttgcatctcaggcctctgggctctggccgggctgggggctattgtccggctgagcggcctgggctgcggcccctccccgtccccgggaccagcctcacccactcgctctgccgcaggtcacacaggccgcctcctgcgggcattgctgggcctcagcctgctcttcctggtggcccatctcgccctccagatctgcctgcatattgtgccccgcctggaccagctcctgggacccagctgtgagtcgctgagggggcggggtagggatagccatcctgggggtcagggagagggccctgcagtgaccccgagtctcctgggggggttgactcagcctgatttatgtctggcctggatggtccaggtgaaacgctccagggatgaccaggccacggtgctggctgggcagagcctgacctgggttcccccgtctttctctgcaggcagccgctgggagaccctctcgcgacacataggggtcacaaggtaagaccattcctcccacccccaaccagcaagcctcccttggggatttcaggccccaggaagtggggggacccaggagggacagagggggacctggagactcatccacactcccacccacacctggagacccatccacgctcccacccacacctggagacccatccacactctcacccgcacctggagacccatccacactcccacccgcacctggagacccatccacactcccacccgcacctgcagacccatccacactctcacccgcacctggagacccatccacactcccacccgcacctggagacccatccacactcccacccgcacctggagacccatccacactcccacccgcacctggagacccatccacactcacacccacacctgtcctcttggtctgaccgcggctgctccctgctctccgcaggctggacctgaaggacatccccaacgccatccggctggtggcccctgacctgggcatcttggtggtctcctctgtctgcctcggcatctgcgggcgccttgcaaggaacacccggcagagcccacatccacgggagctggtgagggcagctgcgtcacccgtgtgtcagggaggtcattcgagagctgtggtctcagccattttgagggttattttaatctttttaaaacagatgtagacgttttggttgtaagttggtgttaaagagaggaggaagttccaaatcccaccccggggcccagcctgcagttccatccgttcagacctgtttctactcgggctctgcctctagtcagaaacctccacgccccgacatggcatctgtgcccttaggaactcttcacaggggaattatttggggccacgcggtggtggaaacctgcagtgctgggcagtgggtctggctggagagccactgcagaagggctgagaaggggcggccccagcaggccccatgcacttacaggcaaacaggctgtcggcccagagccccagcaggggcctggaccccaggagcgacgggcctgaagcagggcctctgtcctcggagtgggaggcagagtgaactttagctgctacaagacttggaggtccggccccgggaatctctgagctatggccccctcacacggacagggcatgagctggggcctgcgacacccaacgttgactgctcagaccctctcgtccctgcctgggcccacacttgccatccccaggctcagccaggatttatggccacctgggtgtcctggcccacgaactccctgccccgggcacccagcctcctcccacctcaccctcccagtgcccactcgtcagatagctgatgctcgccacgcacatgggcctcaacttctagaggagtcccgagagggaaaggggttccccaagccacacggtaggtcgagtacacctgctcagcctgcagaggcggcctgccctcctgcctgtcctgtcgccaccccatggggcaaggctggtgcccgcatttcagaagtgggaacggggacctggaggtcaagtggcttttccgggcatttgagggggtccagcgctcatgaggactgtgctgctgcctgtcctccaagagacccgcccctccctcgtcagcccctccatgctgatgggggcatggggcatgagcttcctagagtttggctgctgggaaggggctgggtggctggactctggtcatcttcatcttggtgtgatgggagcctgagtgtgcagctgcgccctgggcggccagcgtggacagacttcagagtgagctgggggcaccaggcacctgcaggataccgttccgaaagatctagaagccccatgacccggcctggcagggcccccgggcgaccctcactcccacatatgggcaacacacacacgtgtttgcaggcatgcgacggagtgtgccagccagcccgaggggtgctggccgctagaggagggtgaggggcgtctgcagcatggctgcggcacgtggggcccaagtcagggccgggcctgcccctcggtggcctcacaccccaagtcagggccgggcctgcccctcggtggcctcataccccaagtcagggccgggcctgcccctcggtggcctcacacctgttctctcacagcagctgcctctgttctttccactcccagaaatgctcatttttcacggctctgctcagaagatttcccagggccagggccagggccaggctgtgggtggggtgagcgatggcgccctgcgttctgatccagcctcgcccccagctgctgtgtggtctctgagaggggtgggctcagggcctgggcctctcctgccctgatctccctgttaccctggcctcggagcctcccagcagcccctgcagccctggaggcctgtgaggacaggactggtctggttcatctgtcccgtcaagcccccatcccatttgacagaaggggaataggctccgagaggctgtgacttgcctcggcctgcagagcacggagactgtcagagctggggtggggcctatcgtccagccccacagcctgcggtctcagcccgggcacctggcgcctcagccaccaggagagcccccgagtcacagatggggacactgggccggtggcagccaggccagagccagggcatctgacgacagccgtgttctttccaccacacctggctttccctggtgtttgggaaatggctgtgttctgggaaatctttagtcaggtccagggaaaacaggcccggcaggtctctctccccagccccaggccggcctgtcacttcttctaggcagctggccacctctgtccccccagaggcttggggagggcaggagtaccacccctatcttccaggcagagccacccagaaggctggcaggggtacacaggagaccctggagcctggccatgtccctcaggcccccttggtctctagagaccccccggggtatagacagggccccgctgctccctgggtgcgtggttcggggagatgaggtggtataggacagtctgtggtctgtctgtacctggcaaggtcatcacgtgcctgggcttggcaggacagaccctgggtcttcggccagggtgggagctgctaccaggaaggcctgcaggaactgtgagcttgagtgaggaagtaggaaggtgtcaggcagacctcagggacggctggggcctgtgcccggggaggctgtcctgtggccctcagaggagcagctgtggatgtggccgcctcccacgctcctggctgggcaggtgtgggctggagaggtggcgtcagtgcgatacacctgaccttgcccctgtccgtgaccttggcaggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggtacgcaccgaggcagggggcactggcagtcacactgggaggggtcttgggagttccctgatgactgtggagacagcgggacacatggcactggccaggtaccaccctgtgtgcccctgccccgcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctaggtaacggcttgccacacagcccctttttcctgccaccctggtcccgcccacctggctcgtctagcccctgtggccccactgcctctggggtggtaggctgtgacgggtcttctctggacagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccaggtgagcacctgccacccatggtgggtgggctgaggccaggccatggggctggtctcaggacctcctgcctctgggtggggtgtggagctggtttgggctcaagacgctggtctctgcagaggaaggaggcggcaaaggggtatgaggctcgggagctggagctagcagagctggaccagtggccccaggaacgggagtctgaccaggtgagcagccaggcaggtggagacgccagcgtggggggcgcccggccagcccgtgcatggctcagcgctgcttgcccacagcacgtggtgcccacagcacccgacaccgaggctgataactgcatcgtgcacgagctgaccggccagagctccgtcctgcggcggcctggtgagtaccgcacactgcaaggtatggctgggtgcggggggcggggcggaggccggtgctgccccctggtggccgcctggcgctctcgcatgctcgcgccgcacctctgcctgccgccccctcgggggcccaggacatccacgggtcggtgtcagtgacccccgagacccccagggcagccgagtggccatgtcactgaccaacccccaagacccccagggcagctgagtggccgcgtcgttgatccccaagaccccatggggggcctccaggtcccccaacccctccccagagaatgtggctatgctgtcttgtgctgttagctctgggagctgctccaggtggcccagtggccccaggaggccgctcgtccagggcaggggctggcctgggaactctgtgttggccacgtcgccttgggagggcctgggggctctttctggctactttctttctttaccctaacccttgattttccattttgcaatgtgtttctgaatgaagcaaatgaagccacggccctggggtgggggtcctgagagtcttcaggtgcgcagagctggaaagggggtcagggccacctttcccaccctttcaaggaaagtgaggcccagagaacggcaggtgctggcagggccatccctgacgctcagggacggtgtcagcccaattgccggagccctcgtgttctgcccatagcccaccgggggcctgtctctcctgctgtgtgcttgcccagggcccagattttagggcatagtcagggtggggaggcctgcagatcaacctgccgaagctgaccgctgtccccacctgcagtgcggcccaagcgggctgagcccagggaggcgtctccgctccacagcctgggccacctcatcatggaccagagctatgtgtgcgcgctcattgccatgatggtaggcggctgtgggggttggggtgggcggccccctctgccgcgcaggtgtggggcatcgcctgggtggggtgcgctgggcagctgtgcagccccctctgccgcgcaggtgtggggcatcgcctgggtggggtgcgctgggcagctgtgcagccccctctgccgcgcaggtgtggggcatcgcctgggtggggtgcgctgggcagctgtgcagccctctctgccgcgcaggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatggtgagtgtgcaccaccacatccgggggtgcctgggtgcgcagacccatcagggttgtcgtcctgttcaatgtccacttgcccgggggagtggcagcgccaagaaggcagatgtgtctgtctgtccccttctgcccacccagagccagcccagagtagcttctcagtgagcgtttgttgactgaataaacagacaaccttgtgttggcacgggcaccacccctgtgccctgacactgtgtgagcgtgggctctgttggcacgggcaccacccctgtgccccgacactgtgtgtgagcatgggctatgcccattggcacaggcaccacccctgcgcccctgacagtgtgtgtgagcgtgggctctgcccattggcaccatgcacagccctgggtctcagtgacaagctgtgcaggccatgtgttcacagggtgcctgcgtgtccatgtgaaacgggtgccagcatcgtgtctggacacctgtttgcaggccagtgggtctcatcttgtgaaacttgtgagcctgtgtgccaacatatgcacctgtgagcttgtgtgcatatgtgagcacgcatgtgggcaaacatgcactccagcatgtggacatgtgtgcaggtgcgtgcatggatctgtgcccacgtgaactagtgaacccgtgtgtgactctgcgtgtgagcgcaagtgaacccatgcactcatccatggatgtgagagtgtgtgctcgtgtgcctctgagtgggtgtgagcgagagggtgttcggtgcctgtggggaggctgcggtggatgggctggtgccagccgcctgagagctcttgccccctgctataggagggtgctgggtcccccggctgtgggaggggtgctgggccccccggctgactgtgacaccctgcgcttgtcacagttgctctacaccctgaccttctggctcctgctgcgccagtttgtgaaagagaagctgctgaagtgggcagagtctccagctgcgctgacggaggtcaccgtggcagacacaggtgagtggtgggccagaggcgggggttgccctcctgcctgcccgccctgatgccatcgcctgcccctggcttggcccacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtggctggggggccgggatgggggctggggcacggaccctccccgcggtcctcaccacccccacctcacccggcaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagtgagtccaggctggggcggtggggcaggggcgccgaaaccccgtgcacttccccggggctgcagcggctctgccgggggccgggccggtgctgatgctgcccctccacaggctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggtgcggccccgccctccctgtccggccctggagaggtgtagcctcctgggccagggagggagccaggtgggagttggacaggagccacatcttccaccttcagatcccaaggggcatttgctcataccaaggggatggcagtagcgtggaggtcacagggacagtgggcatgagttgcgacacagctgtgcacctgaactggcagctgcagcagaagcggtgccgacagggcttcttccagccccaggaaatgaggggcaggaacccagttgggagatgacattttcggaccctctcccaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggtgggttgggcgggcagagcacagctgccacccagtctgctgtgccatgtcccagctcggggggcgttggcagagtcccctctgggctccagagcctcttcctcacaggggacccgggaatccccgtttgtgccccgcactgaccctcacaccatcacaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgagtgtggcaggcaactcagcttcccatctggggtggggtcgctctggcctgcccagctggcctccccaagcccagccccacgtgcccactgccctccccaagcccagccccacgtgcccactgccctcaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgaggtaccggcccccgagggctgggacgggaggaagctccaggcaactctgtattcgcagcccgaccctcctggggcagctgcctcagtgcagtggggccagcaatggagatggaggactctcccctgggggcgccaagggggcttcctggaggcagcatccttcgacctcaactgtggaccaggggcgcactccctgcacacaagggtgtccagtaggggcggagtcccagggtctccggcagtgaggacgggagggccccacccctggacagggagagacagtcaggcatctctgcctgggaccttctcgcacatccctccttctccctggacctctcttcactcccccagcccctgcccgtggtctccctgtttctcaaacacctggtccccttccccgtgaaggtggctccaaggctggcagcccccgtgtccctggctggggagcagtggacctgccccagagctgtggctgtggtgggctccgggcagggccagggggcactgtggcctgggagggggcactgatgcctggcctcttgccagcccttccccaacagcaccaacttgctgcccacggagatcagccagtccctgctgtaccgggggcccgtggaccctgccaactggtttggggtgcggaaagggttccccaacctgggctacatccaggtgagttgaagggctggtgggcggctgggcgggcgagtacccggctgccccctgacccttgccctccgcagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggaggtgaggcaaggacattgcctccccctggggcagggcttggccttcgggagggagggacggctgcaccgtgcaggcaccgcaagcctggccccacctgggtttgcctgggccacagagggtgggggactcagggccaggcacggcttccctggactcctgtggtgtgtcggtgctgacaacaggcagggggccaagttagatctgctctactgtacagcccacctcctggagcctcagtttcccctgcacgatggcaactgccagccactcctgccctcttgacagcgccgctggccctgtccttgcttgatgcccgcagcctccaggcagggctgctgcaagcctgaggcctgctgggtgggacaagaaagtccctccccccagactcagtgcatccccacaccccgccctctcccctccccagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattggtgaggggcacgtggcttgggtgggagtgggctttgtggctttgtggatgcccgtgggggtgtttcccgcctgccccagactcctgtccaccctcctagacttagccttggcctcctccagtccctcctctctgctccacatcctacccaggcgccatcacctgcatcctgtctctcgggggcggcctggccccctccaggctctgctgttctcttttccttttttgcccagtattctatcttgaaatatttcaaaccttcaggaaagttgtgagtcacacaaggagcactgcgtcctctccgggccccgtgcgggtcggccgtgatgcctcacacccaagtgctgcctcgagcatgcgtctcctgggtgagggtgtctgagcccacagccccacacccgtggtcccgtctcctggcagtgccatcgtcaaacgtgtcctctgcattcaaacagccccggcctcagcacccttctttgtggccatttggttttcagacgggatctggtctgatgtttgctctagtctcttgtcttgggtctaagccgcccccgcctctcctgtctcttgggaagttccggagggaggccggtagcgttgctgacgccgtgagactggatttgcgtggctgtcctggtgctgcaggtctgctcaaggcacacagcaccctgcggtctgagatggggagtcacatttgtgcacgtggccggctcaggggcgtcccacctgccccacagtggcacccagcctgttggcactggtgggctgtgtggggtcagccttgtggttttacgaaacagactttctctctgctgtctctgtgtgtctgtcagctgggattcccatcaaggacagctgccatttgttactggctgctttccaagaattctgtcatccgcagaccctgggcctcccctctgctgagtgggtcctggcccctccggccacacactgttacatcatctccccgtatttggctgggcatggtggttcacgcctgtgatgccagcactttgggaggctgaggcaggagcatcccttgaggccaggagtttgaaaccactctggtcaacatagcaagaaccctttttttttttttttttttttttgagatggagtcttgctctgtcacccaggctggagtacactggcacaatctcggctcactgcagcctccgcctcccgggttcaagcaattctcctgtctcagcctccctcgtggctaggattacaggcaagcaccaccaggcccggctaatttttgtatttttagtagagacggggtttcaccatcttggccaggctggtcttgaactcctgacctcaggtgatccacccgcctcccaaattacaggcctccctcctgggattacaggcgtgagctgccacgcccggccccgtcttgttttctgctcccaggcgctgctgcctcatcttctgctacccaggcccagccttgtgctcacagccattgctccagggagcccaatcgagttctaggagcgtgaggtttagagcccggggtctgggcgctgggtgtgcctgttgctacagggctgcctcagcctctgggccctccagctcttccttgttgaaacatctgctttcgagcatcaccgaggccagctccccgtctcctgtccacctcttccttgttgaaatacctgctatcaagcgtcacctaggccagctccccttcttctgcctccttccacgcggctgcgccatgcagtcgccatcctgtgagatcagcatgtcctgggttccccaacatcgagggtaactttgtttttgtatcgtgaggttccctctgtggcagatggggctgtgggttcagcatgtcctgggttccccagcatcgagggtcactttgtttttgggtcgcgaggttccctctgtggcagatggggctgtgggttcagcatgtcctgggttccccagcatcgagggtcactttgtttttgtgtcgcgaggttccctctgtggcagatggggctgtgggttcagcatgtcctgggttccccagcatcgagggtcactttgtttttgggtcgcgaggttccctctgtggcagatgggggctgtgggttcagaatgtcctgggttccccagcatcgagggtcactttgtttttgggtcacgaggttccctctgtggcagatggggctgtgggttcagcatgtcctgggttccccagcatcgagggtcactttgtttttgtgtcgcgaggttccctctgtggcagatggggctgtgagatcagcatgtcctgggttccccaacatcgagggtcactttgtttttgggtcgcgaggttccctctgtggcagatggggctgtgggttcagcatgtcctgggttccccagcatcgagggtcactttgtttttgggtcgcgaggttccctctgtggcagatggggctgtgggttcagcatgtcctgggttccccagcatcgagggtcactttgtttttgggtcgcgaggttccctctgtggcagatggggctgtgggttcagaatgtcctgggttccccagcatcgagggtcactttgtttttgggtcgcgaggttccctctgtggcagatggggctgtgggttcagcatgtcctgggttccccagcatcgagggtcactttgtttttgggtcacgaggttccctctgtggcagatggggctgtgggttcagcatgtcctgggttccccaacatcgagggtcactttgtttttgggtcgcgaggttccctctgtggcagatggggctgtgagttcagcatgtcctgggttccccagcatggagggtcactttgtttttgtgtcgcaaggttccctctgtggcagatggggctgtgagttcagcatgtcctgggttccccagcatggagggtcactttgtttttgtgtcgcgaggttccctctgtggcagatggggctgtgagttcagcatgtcctgggttccccagcatggagggtcactttgtttttgtgtcgcgaggttccctctgtggcagatggggctgtgagttcagaatgtcctgggttccccagcatcgagggtcactttgtttttgtgtcgcgaggttccctctgtggcagatggggctgtgagatcagcatgtcctgggttccccaacatcgagggtcactttgtttttgggtcgcgaggttccctctgtggcagatggggctgtgggttcagcatgtcctgggttccccagcatcgagggtcactttgcttttgggtcgcgaggttccctctgtggcagatggggctgtgagttcagcatgtcctgggttccccagcatggagggtcactttgtttttgtgtcgcgaggttccctctgtggcagatggggctgtgggtttcgcagatgcgtggagtcacatccatgccctcagtccttagggaccgaccctccctgcctcacacgcctcccaggaagtgtggccgggggccggcagtgccacggctccctccccagcaggccccggccgctcccatccccagcacgtggtcctatcagaacgccacgtcagcaggactcccagcaggtggcctttaggtctggcttctttcacttggcagagcacactgaggtctgtctaggctgtcgcatggatcccggtcccacgtgctgagcagcgcgttcccagctgtggttgctgcaggttgaacttttcctggctgcaggcgtccgtgcagcttctggccgttgttttcagagctgtcctatcacacgcactgtcctatcatggaatatgacgccgtgtgggccacaactcaggcccagcagcccccacccccgtgctctctggcctcctgctcagttcctttgcccccaggggcttggtgcagagttgaaggaatctgtgtgtgtgaacacacaggacactagagctgtcagttctcgagacaccaggtgtgcgcgaggtgattcccatggaccctgagggctggtgatagactcgggtcaacgggtggggaccgggtgtctcaggccccaggcaggcccggcccttcctgacatgacaccccttcccccagattatccctggcgctggagccgggccgtccccatgaactccgcactcatcaagtggctgtacctgcctgatttcttccgggcccccaactccaccaacctcatcagtgagtgccccccaccacccccgcctctgcagaggaccctcagagtacattcacgcccccaaatctgctcacaagtgtgcacacaggcgtgcacgggcggaggtgtggtcaggcacatggcggcctgcaggccctgacctcgcacgcacgcacgcagacctcagcctgtgtgcacggcagcccttgtgcagatgccctcacaccggggctcccccagggacacccggccactcacccaggcagacgtgtgtccgctcccagcggctgcacgccgacaggcctggggtgggaggtgggatttatgcgccgtgcccacctcgtgtgggtccccgtgtggcacagcggcggctcctgtgtcctgcaggcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtgggttccacgtcaccctccacggggaaccttctgggaggggtggccggggcgcccgccctgacgctccggcctggcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcggtgagcctccggcccccccgcacccaccgccctggggccccgctggccccgctgaccctgctctcccccagctcctggcctgcgtcttcgtggagcagatgcagaccggcttctgctgggtcatccagctcttcagccttgtatgcaccgtcaagggctactatgaccgtgagtggccaggacggtggcgggggagggcgtggggaagccccctgctcctgggccctgggcctgacccttgccggtgcctgccttgcagccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggcaagcttgggcccagacacagcccagagctcccgtcttggggctgggagggggcaatgggaggttcctcactgtctcaggccccggcccgtggagggcaggctctgccactctgtgacatgggcgtgtcatctagagggagaatgaaggccggcagatccccggcaccatcacactctgccccagtgctgggtctgtcagagaccacaggctgcagtgctgacggtggctggtgtctcacccccagccaactttcccactaagggctaagtttctccaccagcgggagggccactgtgtggtgtcacgactgccccagggaggggttctggcttggggccagctttgccttcttccctgcagctgtggtggggtgggtgccaccagacgcccctgcatctgtacggcagaagggcctgtcctcgccgcagacagcacggagggtgggggcagcagatgcctcccccgtgggtgcctcttgtccagcgtgggcagagaggagcaggctgagctgtcccgggctgagcggggagcggcggctgcccatgttgctggggtcgagtgcctggtgctcacaccccatccccgcctccctacaggggcttcgccctctacaacgctgccaacctcaagagcattgactttcaccgcaggatagaggagaagtccctggcccagctgaaaagacagtaggtgcctctggggcggggactccccggctcctccccccaatgctcagcataccccacctttccccaccacaggatggagcgtatccgtgccaagcaggagaagcacaggcagggccgggtggaccgcagtcgcccccaggacaccctgggccccaaggaccccggcctggagccaggtgagtgcagctggagtcgggcacccagggccccgtgtccagcatgtctgtgcctgctggcgtgtgctgcgtctgtgcccatgtgacgtcccacagggctcccagcccgcctgtcctgtccgcatgatcaccctctgtctggcaggccccatggccgccctgtgactgtccgtccacgcacatgggctctgagccccatggccccacacggcccccgtcactgtgggtgtccgtgtctgtctccacctatcctgtctccaagacgggagcactcacagccccgacccctcctggtggcttgactgctgcctcatgctcaccctgcccctccacagggcccgacagtccagggggctcctccccgccacggaggcagtggtggcggccctggctggaccacgccacaggtacccccaattaggccgcctgtggccaccctctcaggccctctgtgcccccatctgtcctctgcctggcctgctatcttcccctcccttcccccgactcccaggccctgagcgtcaggacgtgctcaggcctcctgggtcggggggtgcctcactggctgcagacccctgggctgactatgtcctctcctggctatgccccagcccttccaacagtgggagtctcggagcttgccccgatgacacatggtggtcgagcagcgatctcacctgggacccagcagcactgcgttattctgtttttgtttctttttgagatggagtctcgctctgtcactgcaggctggagtgcagtggcatgatctcagctcactgcaatctctacctcccgggttcaagtgatcctcctgcctcagcctcccaagtaactgagactacaagcatgtgccccactccaggccttttttttttttttttggagacggtgtcttgccctgtcgcccaggctacagtgcaatggcgtgattgcggctcactacaacccccacctcccaggttcaaaggattctcctgcctcagcctcccaagtagctgggattacaggtgcccgccaccacgcccagctaattttcgtatttttagtagagatggggtttcaccatattggccaggctggtctcgaacctctgacttcaggtgatccgcccgcctcagcctcccacagtgctgggattacaggcgtcagccaccgtgcccggcctgttctgtttttctaactctcacacagcctcctgggttttccccggtcctctgcagtcggcccactctgcaccccagcccgcgctggctctgctcctcagctgccctgcccacctctgtcttgtcccaccgcgctggcctgtgtcttgtgcctgcactgctcccggctactccgcatgggaagggtggctctcgggccttggcccatgcaggcggagggggtctggctgggagtctccctgcatggaaggctggctctcagtgctgcctgcccacagtcatccactccggggactacttcctgtttgagtccgacagtgaggaagaggaggaggctgttcctgaagacccgaggccgtcggcacagagtgccttccaggtgaggtgggagagccccgtcggccccactccaaccacagagcttgtggtcctggaccagggcagcatagagggtgtcagatgcccccagggcctgggagccgagctcctccacctccagttagcccaccccgccccatccaggcctcccaagtcccatgggaaaccaggctacagggacatgggtcatgtgtagcctgctgccccacggtcttggctctgaccacccaggttctggtggctgcccgtggcctgacctgtgagaccggcccaacacctttgtgctggccgcctggctgtcctgggtccatctttgggcccctggctcttggtgttagaccagcccacccaactcctgaatgggtgggagtcttcccccacagcccctcagggtccccatccgggaggggctcagggacacggaggtccctgggagacacagagcagggatctggatctggcgcccggcttgcccaaccccagcttcccgcctgggtctgatggctcgggaggccgggtcctaacccgggggctggccgacagctggcgtaccaggcatgggtgaccaacgcccaggcggtgctgaggcggcggcagcaggagcaggagcaggcaaggcaggaacaggcaggacagctacccacaggtgagctggggggcgtggggactctgaggggaagccgcgggactgccagtcactcaccagcatcctgtgcccaggaggtggtcccagccaggaggtggagccagcagagggccccgaggaggcagcggcaggtacgtgggcccggggctggggagtgggaggtctctcttggccccacaggctgcccctccagcgccccctcccgccctcccgcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcaggtgagcctgcccgtgcaccacgctcgtccctgctctgcctgactacgcccctgcctgcttaacagcctagtcccgcgcccactgcacgaaaccccgtgtggggacaagagctggacgcagccctgagccccctgctgtgccctgcagggcggcgaagtgcacaggggcgtgctggatcagctgtacacaagccaggccgaggccacgctgccaggccccaccgaggcccccaatgccccaagcaccgtgtccaggtaggtgcgggggtgacccgagccccagctgctgcccctggtgtgtgggcatcgcctagccatccccgaccctcgccattcccttgtaccccaaaggaccgtgggcactttccaccctgaccctccctgtagcctggggtcaggccatagagcaggattctctgtgactcggcttccctccccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggtgggggcgggacgcgcacaacaccagcctcaccatggccctcggggagcagccgaacaggggcaggagactgactgtgaccggcaacagatcgggccgtcatgccttcgggcagtcccagactcccccaaacacgcgggtctccctgtaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgaggtgggccgaggccgcgggggagggggcgcccggcccaccgcgccgtgaccctccccgcgtgctgagccccctcccccacagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctggtgagtgtgagccttggctggcaatgcggggctgggcaggccctctgggcacctgtgctctccaccagggaggcaaggccccctcaccacaccctcccgcccctcagtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcggtataagcgccctgcctcacaacctcctgcctacccagttttctgagtggggctactgcagggagggtctttctcagatgagacggccaagcccagtgcgaggcccacctggatcccaggaaggtgccacttctgagccacagctcccggctctgcctacagagccgtccctgactgctgcccccggggatgctccccacgtgtagggtgactgttggcctgggctggcccctcacagttgccccagacagaggacacagccccagctgtctccttgccagtgacactgggagctttcctgtgctccgtctgcttgtctgtcaaacagggagaatgccagcctcttagggtggtcaggagccatgagccaggcccagtccccagggggcccaggcagaagtcagcttttccctacagaagctgaggacagggaggaagaagagggggaggaagagaaagaggcccccacggggagagagaagaggccaagccgctctggaggaagagtaagggcggccgggcggcggctgcagggcttctgcctgtccctgtgagtgatggcggccgggggcagctggggagtgggggtggggaggcgggtactgggcccaggctgagcgcccccttccgcagggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttggggtgagccaggcccgggacccaaacccagtgtacgcagagctcagcagccacccacatcccctgggcttggctccccctgacctgtgctctcctggccacagaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggtgggcccacgcgtgggggcgctcggtctccaggggcggggcagtgcaggctgggggccctgcggggctgtttctgatggggtccttgacctggccatcccgccccaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggtgagtgcaggtccgccggggtgggggtcacggcccgggcatgagggagcccacctgacgggaaccctggctgtgggcaggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaaggtgcctgggcccagggcgggggccgggacaagggccagggatatgccctctccctaagacagaggcactgctgccacgagaacccgtggtgctggaggccctcccagggctcggagcccatggggacatgaggcgagcccacccactagctgatcacgaggccagtgatcttggcagctgcgagtgagtgctgggcgcagaagtgggcagcggagttggtcctgttccaggcaggctggcagcagagcagggcctggtgcagggaggaccggacagccactgtttgctgcattcttgtttaatggcctttctcagagagaattcgtgcgtcagaccactcccccacgtaaaaagtacaactcaggggtttctagtggattcacagttgagcatctgccttctcaccacttcaaaaagaaaccccgggccgggcacagcggctcacccctatcatcccagcactgtaagagacccaggagggaggtactgctcaaggccaggagttcaagatctgcacggccacaagcgagaccctgcctcagcaaatgtaaaaataaaacaattagctggcaatggcagctcatgcctatggtcccagcactttgggaggccaaaggaggaggatcagttgaggccaggagttttaagaccagcctgggcaacatagtgagtgagactctctctacaaaaaaataaacgttagctgggtgtgatggtgcacacttgtgctcccagctactctggaggctgaggtgggaggatggcttgaggccaggagtacagggctgcattaagcctgatcacaccactgcactccagcttgggcaacagagtgagaccctgtctctaaaaaagtaaaaagaagaaacccagtgcccattggctgtcactcggtttcccctcccctggcccctagtaaccccttgtctaggtcctggttctatgagatttgcctacagtggatatttcatgaaaacaggctcagacagcgtttgtccttttgtaatcagctttccttgctcagcatggtgtctctgaggtccacccacgtggcagtatggccttcctgtttatgggcaaatgatattctgtggcatggatggaccacaacatgctcatctgttgatgggctgttgcccgtgatgctgccaggcacgccggtgtacacgtctgtgtgcccgtgcccctggtcctgtggctgcacgccagggccggaagtgctggtggtggtttccatcatgaggagctgctggttttccatagcagctccaccatctaaggttcccaccaccaacacgaggttgcggtttctccacatcctcaacaacctgttactatgtcttttttgttctggccatgctgctgaacgggtggcttgctgtgggctcttctcagttccctgttgaccgacgctgagcgtcttttcatgtgcttggccatttgtgtatcttctctggggaaatgtctattcaaatcctttctccatagtttagttgggcttttgagataggatctcaggctgaagtgcagtggcatgaccttcactcactgtagcctctgcctcccaggttcaagcgattctcccacctcagcctcccgagtagctgggactacaggtgtgcaccaccaggcctggctagtcttttgtatttttggccaggttcgtctcaaactcctgacctcaaagtgctgggatgacaagtgtgagccgccacacccagcagttgggttgttttttattagagttttttctgtttttctgcagatacattggctagaaatgactgaattggaagaattctctgtattctctggttgctagaaccttatcaattaaaatttgcagaaaatttctccaattctatggactgtctcttaaattttcttggtgtctttggaagcacaaagtatttattttggtaatatctggtttactttgtttccttcgccaaatccaggttcatgaagatttgcccgttttcttctaaaagttctatagttttagctctgaagtttcgctctttgatccactttgaggtaaattttggcacatggtatgaggcaggagttgcgtttcattctcctgcctggggcggtgcctgcaccgtgttgaaaaaggttgtccgttcccactgaacggtcacagctcccttgtctaagatcaacgacccctgaacatgagggttccaactggactcttagttctactccactggcctgtgtctgcccaccattactaccgctgtgccatactgaggtcaggctggggcttttctgggctgctgggtgagctggagaatgcggttgtgtgacccgcaggaagggcagagctgagcgtatgacctgtgtcgttcccctccagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgaggtgagcatgtgtgggtccgcctgtccattcccatcccctgggggttctggccaaggtggtgcaccacccccagccgctcctccacgctcatcttcgtggccccgtgtccccgtgcctgccccagccgctgttcaccatgagcgcccagcagccgtccatcatccccttcacggcccaggcctatgaggagctgtcccggcagtttgacccccagccggtaagtggcctctgccctgtgaaagctggtgtggggaggcggctgcagtcactgagggtgtcacttgtacccagctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagaggttcgtcctggacttggggcagtgcctgggtgggtggacccactacagtgggtcacgctgtgttcccacccccagggacctggcgaagggaggcactgtggagtatgccaacgagaagcacatgctggccctggcccccaacagcactgcacggcggcagctggccagcctgctcgagggcacctcggaccagtctgtgtgagtgaagggcccgggtggtgggcaggagggctgtgccaggttggctgggccaggcctgacctgccagcacctccctgcagggtcatccctaatctcttccccaagtacatccgtgcccccaacgggcccgaagccaaccctgtgaagcagctgcagcccagtgagtatgggcgtgggggttgggggaggctagagaggggtgacctgcggcctcaacgatcttctccctccatcccagatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacgggtgagtgagtggctgggggggcaccccgcagctcggggggctccgggcggccccaggactcaccagcttcccccgcagcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaatcaga

SEQ ID NO: 2 - RNA, 8089 nt, Homo sapiens
  source 1..8089: mol_type = mRNA
gggagccgccgtccggcccagctcggccccagtgagccgagcgctgcgctccgccgaggggcagggcggtcgcctgagcgagcgcgggcccgggacgtcggcaccggcggggcggccgaaggagaaggaggaagaggagaaggcggcgcgcgggtccccgcgggtcagccatggcgcgccggccccggggcccccgcaccgccccatagcgccgcggcgtccgctcggtctgggccgggccctgggccctccagccatggagccgcacgtgctcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatattgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaatcaga

SEQ ID NO: 3 - DNA, 8089 bp, Homo sapiens
  source 1..8089: mol_type = other DNA
gggagccgccgtccggcccagctcggccccagtgagccgagcgctgcgctccgccgaggggcagggcggtcgcctgagcgagcgcgggcccgggacgtcggcaccggcggggcggccgaaggagaaggaggaagaggagaaggcggcgcgcgggtccccgcgggtcagccatggcgcgccggccccggggcccccgcaccgccccatagcgccgcggcgtccgctcggtctgggccgggccctgggccctccagccatggagccgcacgtgctcggcgcggtcctgtactggctgctgctgccctgcgcgctgctggctgcctgcctgctccgcttcagcggactctcgctggtctacctgctcttcctgctgctgctgccctggttccccggccccacccgatgcggcctccaaggtcacacaggccgcctcctgcgggcattgctgggcctcagcctgctcttcctggtggcccatctcgccctccagatctgcctgcatattgtgccccgcctggaccagctcctgggacccagctgcagccgctgggagaccctctcgcgacacataggggtcacaaggctggacctgaaggacatccccaacgccatccggctggtggcccctgacctgggcatcttggtggtctcctctgtctgcctcggcatctgcgggcgccttgcaaggaacacccggcagagcccacatccacgggagctggatgatgatgagagggatgtggatgccagcccgacggcagggctgcaggaagcagcaacgctggcccctacacggaggtcacggctggccgctcgtttccgagtcacggcccactggctgctggtggcggctgggcgggtcctggccgtaacactgcttgcactggcaggcatcgcccacccctcggccctctccagtgtctacctgctgctcttcctggccctctgcacctggtgggcctgccactttcccatcagcactcggggcttcagcagactctgcgtcgcggtggggtgcttcggcgccggccatctcatctgcctctactgctaccagatgcccttggcacaggctctgctcccgcctgccggcatctgggctagggtgctgggtctcaaggacttcgtgggtcccaccaactgctccagcccccacgcgctggtcctcaacaccggcctggactggcctgtgtatgccagccccggcgtcctcctgctgctgtgctacgccacggcctctctgcgcaagctccgcgcgtaccgcccctccggccagaggaaggaggcggcaaaggggtatgaggctcgggagctggagctagcagagctggaccagtggccccaggaacgggagtctgaccagcacgtggtgcccacagcacccgacaccgaggctgataactgcatcgtgcacgagctgaccggccagagctccgtcctgcggcggcctgtgcggcccaagcgggctgagcccagggaggcgtctccgctccacagcctgggccacctcatcatggaccagagctatgtgtgcgcgctcattgccatgatggtatggagcatcacctaccacagctggctgaccttcgtactgctgctctgggcctgcctcatctggacggtgcgcagccgccaccaactggccatgctgtgctcgccctgcatcctgctgtatgggatgacgctgtgctgcctacgctacgtgtgggccatggacctgcgccctgagctgcccaccaccctgggccccgtcagcctgcgccagctggggctggagcacacccgctacccctgtctggaccttggtgccatgttgctctacaccctgaccttctggctcctgctgcgccagtttgtgaaagagaagctgctgaagtgggcagagtctccagctgcgctgacggaggtcaccgtggcagacacagagcccacgcggacgcagacgctgttgcagagcctgggggagctggtgaagggcgtgtacgccaagtactggatctatgtgtgtgctggcatgttcatcgtggtcagcttcgccggccgcctcgtggtctacaagattgtctacatgttcctcttcctgctctgcctcaccctcttccaggtctactacagcctgtggcggaagctgctcaaggccttctggtggctcgtggtggcctacaccatgctggtcctcatcgccgtctacaccttccagttccaggacttccctgcctactggcgcaacctcactggcttcaccgacgagcagctgggggacctgggcctggagcagttcagcgtgtccgagctcttctccagcatcctggtgcccggcttcttcctcctggcctgcatcctgcagctgcactacttccacaggcccttcatgcagctcaccgacatggagcacgtgtccctgcctggcacgcgcctcccgcgctgggctcacaggcaggatgcagtgagtgggaccccactgctgcgggaggagcagcaggagcatcagcagcagcagcaggaggaggaggaggaggaggaggactccagggacgaggggctgggcgtggccactccccaccaggccacgcaggtgcctgaaggggcagccaagtggggcctggtggctgagcggctgctggagctggcagccggcttctcggacgtcctctcacgcgtgcaggtgttcctgcggcggctgctggagcttcacgttttcaagctggtggccctgtacaccgtctgggtggccctgaaggaggtgtcggtgatgaacctgctgctggtggtgctgtgggccttcgccctgccctacccacgcttccggcccatggcctcctgcctgtccaccgtgtggacctgcgtcatcatcgtgtgtaagatgctgtaccagctcaaggttgtcaacccccaggagtattccagcaactgcaccgagcccttccccaacagcaccaacttgctgcccacggagatcagccagtccctgctgtaccgggggcccgtggaccctgccaactggtttggggtgcggaaagggttccccaacctgggctacatccagaaccacctgcaagtgctgctgctgctggtattcgaggccatcgtgtaccggcgccaggagcactaccgccggcagcaccagctggccccgctgcctgcccaggccgtgtttgccagcggcacccgccagcagctggaccaggatctgctcggctgcctcaagtacttcatcaacttcttcttctacaaattcgggctggagatctgcttcctgatggccgtgaacgtgatcgggcagcgcatgaactttctggtgaccctgcacggttgctggctggtggccatcctcacccgcaggcaccgccaggccattgcccgcctctggcccaactactgcctcttcctggcgctgttcctgctgtaccagtacctgctgtgcctggggatgcccccggccctgtgcattgattatccctggcgctggagccgggccgtccccatgaactccgcactcatcaagtggctgtacctgcctgatttcttccgggcccccaactccaccaacctcatcagcgactttctcctgctgctgtgcgcctcccagcagtggcaggtgttctcagctgagcgcacagaggagtggcagcgcatggctggcgtcaacaccgaccgcctggagccgctgcggggggagcccaaccccgtgcccaactttatccactgcaggtcctaccttgacatgctgaaggtggccgtcttccgatacctgttctggctggtgctggtggtggtgtttgtcacgggggccacccgcatcagcatcttcgggctgggctacctgctggcctgcttctacctgctgctcttcggcacggccctgctgcagagggacacacgggcccgcctcgtgctgtgggactgcctcattctgtacaacgtcaccgtcatcatctccaagaacatgctgtcgctcctggcctgcgtcttcgtggagcagatgcagaccggcttctgctgggtcatccagctcttcagccttgtatgcaccgtcaagggctactatgaccccaaggagatgatggacagagaccaggactgcctgctgcctgtggaggaggctggcatcatctgggacagcgtctgcttcttcttcctgctgctgcagcgccgcgtcttccttagccattactacctgcacgtcagggccgacctccaggccaccgccctgctagcctccaggggcttcgccctctacaacgctgccaacctcaagagcattgactttcaccgcaggatagaggagaagtccctggcccagctgaaaagacagatggagcgtatccgtgccaagcaggagaagcacaggcagggccgggtggaccgcagtcgcccccaggacaccctgggccccaaggaccccggcctggagccagggcccgacagtccagggggctcctccccgccacggaggcagtggtggcggccctggctggaccacgccacagtcatccactccggggactacttcctgtttgagtccgacagtgaggaagaggaggaggctgttcctgaagacccgaggccgtcggcacagagtgccttccagctggcgtaccaggcatgggtgaccaacgcccaggcggtgctgaggcggcggcagcaggagcaggagcaggcaaggcaggaacaggcaggacagctacccacaggaggtggtcccagccaggaggtggagccagcagagggccccgaggaggcagcggcaggccggagccatgtggtgcagagggtgctgagcacggcgcagttcctgtggatgctggggcaggcgctagtggatgagctgacacgctggctgcaggagttcacccggcaccacggcaccatgagcgacgtgctgcgggcagagcgctacctcctcacacaggagctcctgcagggcggcgaagtgcacaggggcgtgctggatcagctgtacacaagccaggccgaggccacgctgccaggccccaccgaggcccccaatgccccaagcaccgtgtccagtgggctgggcgcggaggagccactcagcagcatgacagacgacatgggcagccccctgagcaccggctaccacacgcgcagtggcagtgaggaggcagtcaccgaccccggggagcgtgaggctggtgcctctctgtaccagggactgatgcggacggccagcgagctgctcctggacaggcgcctgcgcatcccagagctggaggaggcagagctgtttgcggaggggcagggccgggcgctgcggctgctgcgggccgtgtaccagtgtgtggccgcccactcggagctgctctgctacttcatcatcatcctcaaccacatggtcacggcctccgccggctcgctggtgctgcccgtgctcgtcttcctgtgggccatgctgtcgatcccgaggcccagcaagcgcttctggatgacggccatcgtcttcaccgagatcgcggtggtcgtcaagtacctgttccagtttgggttcttcccctggaacagccacgtggtgctgcggcgctacgagaacaagccctacttcccgccccgcatcctgggcctggagaagactgacggctacatcaagtacgacctggtgcagctcatggcccttttcttccaccgctcccagctgctgtgctatggcctctgggaccatgaggaggactcaccatccaaggagcatgacaagagcggcgaggaggagcagggagccgaggaggggccaggggtgcctgcggccaccaccgaagaccacattcaggtggaagccagggtcggacccacggacgggaccccagaaccccaagtggagctcaggccccgtgatacgaggcgcatcagtctacgttttagaagaaggaagaaggagggcccagcacggaaaggagcggcagccatcgaagctgaggacagggaggaagaagagggggaggaagagaaagaggcccccacggggagagagaagaggccaagccgctctggaggaagagtaagggcggccgggcggcggctgcagggcttctgcctgtccctggcccagggcacatatcggccgctacggcgcttcttccacgacatcctgcacaccaagtaccgcgcagccaccgacgtctatgccctcatgttcctggctgatgttgtcgacttcatcatcatcatttttggcttctgggcctttgggaagcactcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccaggtggcgctggtgctggccatccacctatggatgttcttcatcctgcccgccgtcactgagaggatgttcaaccagaatgtggtggcccagctctggtacttcgtgaagtgcatctacttcgccctgtccgcctaccagatccgctgcggctaccccacccgcatcctcggcaacttcctcaccaagaagtacaatcatctcaacctcttcctcttccaggggttccggctggtgccgttcctggtggagctgcgggcagtgatggactgggtgtggacggacaccacgctgtccctgtccagctggatgtgtgtggaggacatctatgccaacatcttcatcatcaaatgcagccgagagacagagaagaaatacccgcagcccaaagggcagaagaagaagaagatcgtcaagtacggcatgggtggcctcatcatcctcttcctcatcgccatcatctggttcccactgctcttcatgtcgctggtgcgctccgtggttggggttgtcaaccagcccatcgatgtcaccgtcaccctgaagctgggcggctatgagccgctgttcaccatgagcgcccagcagccgtccatcatccccttcacggcccaggcctatgaggagctgtcccggcagtttgacccccagccgctggccatgcagttcatcagccagtacagccctgaggacatcgtcacggcgcagattgagggcagctccggggcgctgtggcgcatcagtccccccagccgtgcccagatgaagcgggagctctacaacggcacggccgacatcaccctgcgcttcacctggaacttccagagggacctggcgaagggaggcactgtggagtatgccaacgagaagcacatgctggccctggcccccaacagcactgcacggcggcagctggccagcctgctcgagggcacctcggaccagtctgtggtcatccctaatctcttccccaagtacatccgtgcccccaacgggcccgaagccaaccctgtgaagcagctgcagcccaatgaggaggccgactacctcggcgtgcgtatccagctgcggagggagcagggtgcgggggccaccggcttcctcgaatggtgggtcatcgagctgcaggagtgccggaccgactgcaacctgctgcccatggtcattttcagtgacaaggtcagcccaccgagcctcggcttcctggctggctacggcatcatggggctgtacgtgtccatcgtgctggtcatcggcaagttcgtgcgcggattcttcagcgagatctcgcactccattatgttcgaggagctgccgtgcgtggaccgcatcctcaagctctgccaggacatcttcctggtgcgggagactcgggagctggagctggaggaggagttgtacgccaagctcatcttcctctaccgctcaccggagaccatgatcaagtggactcgtgagaaggagtaggagctgctgctggcgcccgagagggaaggagccggcctgctgggcagcgtggccacaaggggcggcactcctcaggccgggggagccactgccccgtccaaggccgccagctgtgatgcatcctcccggcctgcctgagccctgatgctgctgtcagagaaggacactgcgtccccacggcctgcgtggcgctgccgtcccccacgtgtactgtagagttttttttttaattaaaaaatgttttatttatacaaatggacaatcaga

SEQ ID NO: 4 - protein, 2521 aa, Homo sapiens
MEPHVLGAVLYWLLLPCALLAACLLRFSGLSLVYLLFLLLLPWFPGPTRCGLQGHTGRLLRALLGLSLLFLVAHLALQICLHIVPRLDQLLGPSCSRWETLSRHIGVTRLDLKDIPNAIRLVAPDLGILVVSSVCLGICGRLARNTRQSPHPRELDDDERDVDASPTAGLQEAATLAPTRRSRLAARFRVTAHWLLVAAGRVLAVTLLALAGIAHPSALSSVYLLLFLALCTWWACHFPISTRGFSRLCVAVGCFGAGHLICLYCYQMPLAQALLPPAGIWARVLGLKDFVGPTNCSSPHALVLNTGLDWPVYASPGVLLLLCYATASLRKLRAYRPSGQRKEAAKGYEARELELAELDQWPQERESDQHVVPTAPDTEADNCIVHELTGQSSVLRRPVRPKRAEPREASPLHSLGHLIMDQSYVCALIAMMVWSITYHSWLTFVLLLWACLIWTVRSRHQLAMLCSPCILLYGMTLCCLRYVWAMDLRPELPTTLGPVSLRQLGLEHTRYPCLDLGAMLLYTLTFWLLLRQFVKEKLLKWAESPAALTEVTVADTEPTRTQTLLQSLGELVKGVYAKYWIYVCAGMFIVVSFAGRLVVYKIVYMFLFLLCLTLFQVYYSLWRKLLKAFWWLVVAYTMLVLIAVYTFQFQDFPAYWRNLTGFTDEQLGDLGLEQFSVSELFSSILVPGFFLLACILQLHYFHRPFMQLTDMEHVSLPGTRLPRWAHRQDAVSGTPLLREEQQEHQQQQQEEEEEEEDSRDEGLGVATPHQATQVPEGAAKWGLVAERLLELAAGFSDVLSRVQVFLRRLLELHVFKLVALYTVWVALKEVSVMNLLLVVLWAFALPYPRFRPMASCLSTVWTCVIIVCKMLYQLKVVNPQEYSSNCTEPFPNSTNLLPTEISQSLLYRGPVDPANWFGVRKGFPNLGYIQNHLQVLLLLVFEAIVYRRQEHYRRQHQLAPLPAQAVFASGTRQQLDQDLLGCLKYFINFFFYKFGLEICFLMAVNVIGQRMNFLVTLHGCWLVAILTRRHRQAIARLWPNYCLFLALFLLYQYLLCLGMPPALCIDYPWRWSRAVPMNSALIKWLYLPDFFRAPNSTNLISDFLLLLCASQQWQVFSAERTEEWQRMAGVNTDRLEPLRGEPNPVPNFIHCRSYLDMLKVAVFRYLFWLVLVVVFVTGATRISIFGLGYLLACFYLLLFGTALLQRDTRARLVLWDCLILYNVTVIISKNMLSLLACVFVEQMQTGFCWVIQLFSLVCTVKGYYDPKEMMDRDQDCLLPVEEAGIIWDSVCFFFLLLQRRVFLSHYYLHVRADLQATALLASRGFALYNAANLKSIDFHRRIEEKSLAQLKRQMERIRAKQEKHRQGRVDRSRPQDTLGPKDPGLEPGPDSPGGSSPPRRQWWRPWLDHATVIHSGDYFLFESDSEEEEEAVPEDPRPSAQSAFQLAYQAWVTNAQAVLRRRQQEQEQARQEQAGQLPTGGGPSQEVEPAEGPEEAAAGRSHVVQRVLSTAQFLWMLGQALVDELTRWLQEFTRHHGTMSDVLRAERYLLTQELLQGGEVHRGVLDQLYTSQAEATLPGPTEAPNAPSTVSSGLGAEEPLSSMTDDMGSPLSTGYHTRSGSEEAVTDPGEREAGASLYQGLMRTASELLLDRRLRIPELEEAELFAEGQGRALRLLRAVYQCVAAHSELLCYFIIILNHMVTASAGSLVLPVLVFLWAMLSIPRPSKRFWMTAIVFTEIAVVVKYLFQFGFFPWNSHVVLRRYENKPYFPPRILGLEKTDGYIKYDLVQLMALFFHRSQLLCYGLWDHEEDSPSKEHDKSGEEEQGAEEGPGVPAATTEDHIQVEARVGPTDGTPEPQVELRPRDTRRISLRFRRRKKEGPARKGAAAIEAEDREEEEGEEEKEAPTGREKRPSRSGGRVRAAGRRLQGFCLSLAQGTYRPLRRFFHDILHTKYRAATDVYALMFLADVVDFIIIIFGFWAFGKHSAATDITSSLSDDQVPEAFLVMLLIQFSTMVVDRALYLRKTVLGKLAFQVALVLAIHLWMFFILPAVTERMFNQNVVAQLWYFVKCIYFALSAYQIRCGYPTRILGNFLTKKYNHLNLFLFQGFRLVPFLVELRAVMDWVWTDTTLSLSSWMCVEDIYANIFIIKCSRETEKKYPQPKGQKKKKIVKYGMGGLIILFLIAIIWFPLLFMSLVRSVVGVVNQPIDVTVTLKLGGYEPLFTMSAQQPSIIPFTAQAYEELSRQFDPQPLAMQFISQYSPEDIVTAQIEGSSGALWRISPPSRAQMKRELYNGTADITLRFTWNFQRDLAKGGTVEYANEKHMLALAPNSTARRQLASLLEGTSDQSVVIPNLFPKYIRAPNGPEANPVKQLQPNEEADYLGVRIQLRREQGAGATGFLEWWVIELQECRTDCNLLPMVIFSDKVSPPSLGFLAGYGIMGLYVSIVLVIGKFVRGFFSEISHSIMFEELPCVDRILKLCQDIFLVRETRELELEEELYAKLIFLYRSPETMIKWTREKE